(19) United States
(12) Patent Application Publication
(10) Pub. No.: US 2015/0080570 A1
(43) Pub. Date: Mar. 19, 2015

(54) ALPHA,BETA-UNSATURATED MONOMERS CAPABLE OF MULTIMERIZATION IN AN AQUEOUS SOLUTION, AND METHODS OF USING SAME

(71) Applicants: Coferon, Inc., Stony Brook, NY (US); Cornell University, Ithaca, NY (US)

(72) Inventors: Lee Daniel Arnold, Mount Sinai, NY (US); Maneesh Pingle, Huntington, NY (US)

(73) Assignees: Coferon, Inc., Stony Brook, NY (US); Cornell University, Ithaca, NY (US)

(21) Appl. No.: 14/251,028

(22) Filed: Apr. 11, 2014

Related U.S. Application Data
(60) Provisional application No. 61/810,920, filed on Apr. 11, 2013.

(51) Int. Cl. C07D 417/06; C07D 263/06; C07D 413/06; C07D 239/10

(52) U.S. Cl. CPC C07D 417/06; C07D 239/10; C07D 263/06; C07D 413/06
USPC 544/133; 544/311; 544/137

(57) ABSTRACT

Described herein are monomers capable of forming a biologically useful multimer when in contact with one, two, three or more other monomers in an aqueous media. In one aspect, such monomers may be capable of binding to another monomer in an aqueous media (e.g. in vivo) to form a multimer, (e.g. a dimer). Contemplated monomers may include a ligand moiety, a linker element, and a connector element that joins the ligand moiety and the linker element. In an aqueous media, such contemplated monomers may join together via each linker element and may thus be capable of modulating one or more biomolecules substantially simultaneously, e.g., modulate two or more binding domains on a protein or on different proteins.

### RELATED APPLICATIONS

This application claims priority to U.S. Ser. No. 61/810,920, filed Apr. 11, 2013, which is hereby incorporated by reference in its entirety.

### BACKGROUND

Current drug design and drug therapies have not addressed the urgent need for therapies that interact with extended areas or multiple domains of biomolecules such as proteins. For example, there is an urgent need for therapies that are capable of, e.g., modulating protein-protein interactions, e.g., by modulating, simultaneously, domains on a single protein or both a domain on one protein and a domain on another protein. There is also an urgent need for such therapies that modulate fusion proteins, such as those that occur in cancer.

For example, signaling pathways are used by cells to generate biological responses to external or internal stimuli. A few thousand gene products control both ontogeny/development of higher organisms and sophisticated behavior by their many different cell types. These gene products can work in different combinations to achieve their goals and often do so through protein-protein interactions. Such proteins possess modular protein domains that recognize, bind, and/or modify certain motifs. Protein-protein and protein-nucleic acid recognition often function through protein interactions domains, for example, such as the SH2, SH3, and PDZ domains. These protein-interaction domains may represent a meaningful area for developing targeted therapies. Other macromolecular interactions that may serve as potential targets for effective therapies include protein-nucleic acid interactions, protein-carbohydrate interactions, and protein-lipid interactions.

Current drug design and drug therapy approaches do not address this urgent need to find drugs that interfere with intracellular protein-protein interactions or protein signaling. Although antibodies and other biological therapeutic agents may have sufficient specificity to distinguish among closely related protein surfaces, factors such as their high molecular weight prevent oral administration and uptake of the antibodies. Conversely, orally active pharmaceuticals are generally too small to disrupt protein-protein surface interactions, which can be much larger than the orally active pharmaceuticals. Further, previous attempts to link, e.g., two pharmacophores that each interact with e.g. different protein domains have focused on large covalently linked compounds assembled in organic solvents. These assemblies typically have a molecular weight too large for oral administration or effective cellular and tissue permeation.

### SUMMARY

Described herein are monomers capable of forming a biologically useful multimer when in contact with one, two, three or more other monomers in an aqueous media. In one aspect, such monomers may be capable of binding to another monomer in an aqueous media (e.g. in vivo) to form a multimer, (e.g. a dimer). Contemplated monomers may include a ligand moiety (e.g. a pharmacophore for the target biomolecule), a linker element, and a connector element that joins the ligand moiety and the linker element. In an aqueous media, such contemplated monomers may join together via each linker element and may thus be capable of modulating one or more biomolecules substantially simultaneously, e.g., modulate two or more binding domains on a protein or on different proteins.

In one aspect, a first monomer capable of forming a biologically useful multimer when in contact with one, two or more second monomers in an aqueous media is provided. The first monomer is represented by the formula:

X¹—Y¹—Z¹ (Formula I)

and pharmaceutically acceptable salts, stereoisomers, metabolites and hydrates thereof, wherein
X¹ is a first ligand moiety capable of binding to and modulating a first target biomolecule;
Y¹ is absent or is a connector moiety covalently bound to X¹ and Z¹;
Z¹ is an activated π-moiety; and

the second monomer has a nucleophile moiety capable of binding with the Z¹ moiety of Formula I to form the multimer.

In another aspect, a method of administering a pharmaceutically effective amount of a multimer compound to a patient in need thereof is provided. The method comprises administering to the patient thereof an amount of the first monomer and an amount of the nucleophile monomer in amounts effective such that the pharmaceutically effective amount of the resulting multimer is formed in an aqueous media.

In yet another aspect, a therapeutic multimer compound formed from the multimerization in an aqueous media of a first monomer is provided. The first monomer is represented by:

X¹—Y¹—Z¹ (Formula I)

and pharmaceutically acceptable salts, stereoisomers, metabolites and hydrates thereof,

and a second monomer represented by:

X²—Y²—Z² (Formula II)

and pharmaceutically acceptable salts, stereoisomers, metabolites and hydrates thereof

In still another aspect, a method of modulating two or more target biomolecules substantially simultaneously is provided. The method comprises contacting an aqueous composition comprising said bimolecular target with a first monomer represented by:

X¹—Y¹—Z¹ (Formula I)
and pharmaceutically acceptable salts, stereoisomers, metabolites and hydrates thereof, wherein
X¹ is a first ligand moiety capable of binding to and modulating a first target biomolecule; and
   a second monomer represented by:

X²—Y²—Z² (Formula II),
and pharmaceutically acceptable salts, stereoisomers, metabolites and hydrates thereof, wherein
X₂ is a ligand moiety capable of binding to and modulating a second target biomolecule;

wherein upon contact with the aqueous composition, said first monomer and said second monomer forms a multimer that binds to the first target biomolecule and the second target biomolecule.

In yet another aspect, a method of treating a disease associated with two or more target biomolecules in a patient in need thereof is provided. The method comprises administering to said patient a first monomer represented by:

X¹—Y¹—Z¹ (Formula I)

and pharmaceutically acceptable salts, stereoisomers, metabolites and hydrates thereof, wherein

X¹ is a first ligand moiety capable of binding to and modulating a first target biomolecule; and

administering to said patient a second monomer represented by:

X²—Y²—Z² (Formula II), wherein

X² is a second ligand moiety capable of binding to and modulating a second target biomolecule, wherein upon administration, said first monomer and said second monomer forms a multimer in vivo that binds to the first target biomolecule and the second target biomolecule.

### DETAILED DESCRIPTION

Described herein are monomers capable of forming a biologically useful multimer when in contact with one, two, three or more other monomers in an aqueous media. In one aspect, such monomers may be capable of binding to another monomer in an aqueous media (e.g. in vivo) to form a multimer, (e.g. a dimer). Contemplated monomers may include a ligand moiety (e.g. pharmacophore moiety), a linker element, and a connector element that joins the ligand moiety and the linker element. In an aqueous media, such contemplated monomers may join together via each linker element and may thus be capable of modulating one or more biomolecules substantially simultaneously, e.g., modulate two or more binding domains on a protein or on different proteins. For example, contemplated monomers may be separate or separatable in a solid or in an aqueous media under one set of conditions, and when placed in an aqueous media having one or more biomolecules, with another (e.g., under a different set of conditions), can 1) form a multimer through the linker on each monomer; and either: 2a) bind to the biomolecule in two or more locations (e.g. protein domains) through each ligand moiety of the respective monomer or 2b) bind to two or more biomolecules through each ligand moiety of the respective monomer. In an exemplary embodiment, disclosed monomers may interact with another appropriate monomer (i.e. a monomeric pair) in an aqueous media (e.g., in vivo) to form a multimer (e.g. a dimer) that can bind to two separate target biomolecule domains (e.g. protein domains).

The ligand moiety of a contemplated monomer, in some cases, may be a pharmacophore or a ligand moiety that is e.g., capable of binding to a biomolecule, such as for example, a protein, e.g. a specific protein domain, a component of a biological cell such as ribosome (composed of proteins and nucleic acids), or an enzyme active site (e.g. a protease, such as tryptase). In some embodiments, the linker element comprises a functional group capable of forming a chemical bond with another linker element. In some embodiments, the linker moiety may also serve as a signaling entity or “reporter,” and in some instances the assembly of two or more linkers can produce a fluorescent entity or fluorophore with properties distinct from the individual linker moiety. In another aspect, a plurality of monomers, each comprising a linker element, may react to form a multimer connected by the linker elements. In some embodiments, the multimer may be formed in vivo. In some instances, the multimer may have enhanced properties relative to the monomers that form the multimer. For example, in certain embodiments, the multimer may bind to a target with greater affinity than any of the monomers that form the multimer. Also described are methods of making the compositions and methods of administering the compositions.

In some embodiments, a plurality of monomers may assemble to form a multimer. The multimer may be used for a variety of purposes. For example, in some instances, the multimer may be used to perturb a biological system. As described in more detail below, in some embodiments, the multimer may bind to a target biomolecule, such as a protein, nucleic acid, or polysaccharide. In certain embodiments, the multimer may be used as a pharmaceutical.

Advantageously, in some embodiments, the multimer may form in vivo upon administration of suitable monomers to a subject. Also advantageously, the multimer may be capable of interacting with a relatively large target site as compared to the individual monomers that form the multimer. For example, a target may comprise, in some embodiments, two protein domains separated by a distance such that a multimer, but not a monomer, may be capable of binding to both domains essentially simultaneously. In some embodiments, contemplated multimers may bind to a target with greater affinity as compared to a monomer binding affinity alone.

In some embodiments, a contemplated multimer may advantageously exhibit enhanced properties relative to the monomers that form the multimer. As discussed above, a multimer may have improved binding properties as compared to the monomers alone. In some embodiments, a multimer may have improved signaling properties. For example, in some cases, the fluorescent properties of a multimer may be different as compared to a monomer. As discussed in more detail below, in some embodiments the fluorescent brightness of a multimer at a particular wavelength may be greater than the fluorescent brightness at the same wavelength of the monomers that form the multimer. Advantageously, in some embodiments, a difference in signaling properties between the multimer and the monomers that form the multimer may be used to detect formation of the multimer. In some embodiments, detection of the formation of the multimer may be used to screen monomers, as discussed in more detail below. Also as discussed in more detail below, in some embodiments, the multimers may be used for imaging or as diagnostic agents.

It should be understood that a multimer, as used herein, may be a homomultimer (i.e., a multimer formed from two or more essentially identical monomers) or may be a heteromultimer (i.e., a multimer formed from two or more substantially different monomers). In some embodiments, a contemplated multimer may comprise 2 to about 10 monomers, for example, a multimer may be a dimer, a trimer, a tetramer, or a pentamer.

In some embodiments, a monomer may comprise a ligand moiety, a linker element, and a connector element that associates the ligand moiety with the linker element. In some embodiments, the linker element of a first monomer may combine with the linker element of a second monomer. In some cases, the linker element may comprise a functional group that can react with a functional group of another linker element to form a bond linking the monomers. In some embodiments, the linker element of a first monomer may be substantially the same as the linker element of a second monomer. In some embodiments, the linker element of a first monomer may be substantially different than the linker element of a second monomer.

In some cases, the ligand moiety may be a pharmacophore. In some embodiments, the ligand moiety (e.g., a pharmacophore) may bind to a target molecule with a dissociation constant of less than 1 mM, in some embodiments less than 500 microM, in some embodiments less than 300 microM, in some embodiments less than 100 microM, in some embodiments less than 10 microM, in some embodiments less than 1 microM, in some embodiments less than 100 nM, in some embodiments less than 10 nM, and in some embodiments less than 1 nM.

In some embodiments, the apparent IC₅₀ of the first monomer against a first target biomolecule and the apparent IC₅₀ of the second monomer against a second target biomolecule may be greater than the apparent IC₅₀ of a combination of the monomers against the first target biomolecule and the second target biomolecule. The combination of monomers may be any suitable ratio. For example, the ratio of the first monomer to the second monomer may be between 10:1 to 1:10, in some embodiments between 5:1 and 1:5, and in some embodiments between 2:1 and 1:2. In some cases, the ratio of the first monomer to the second monomer may be essentially 1:1. In some instances, the ratio of the smaller of the apparent IC₅₀ of the first monomer and the second monomer to the apparent IC₅₀ of the multimer may be at least 3.0. In other instances, the ratio of the smaller apparent IC₅₀ of the first monomer or the second monomer to the apparent IC₅₀ of the multimer may be at least 10.0. In some embodiments, the ratio of the smaller apparent IC₅₀ of the first monomer or the second monomer to the apparent IC₅₀ of the multimer may be at least 30.0.

For example, for disclosed monomers forming a heteromultimer, the apparent IC₅₀ resulting from an essentially equimolar combination of monomers against the first target biomolecule and the second target biomolecule is at least about 3 to 10 fold lower, at least about 10 to 30 fold lower, at least about 30 fold lower, or at least about 40 to 50 fold lower than the lowest of the apparent IC₅₀ of the second monomer against the second target biomolecule or the IC₅₀ of the first monomer against the first target biomolecule.

It will be appreciated that for monomers forming homodimers (or homo-oligomeric or homomultimeric, as described below), in aqueous solution, there may an equilibrium between the monomeric and dimeric (or oligomeric) states with higher concentrations favoring greater extent of dimer formation. As the binding of monomers to the target biomolecule increases their proximity and effectively increases their local concentration on the target, the rate and extent of dimerization (oligomerization) is promoted when geometries are favorable. As a result, the occupancy of the target by favorable monomers maybe nearly completely in the homodimeric (or oligomeric) state. In this manner the target for example, may serve as a template for the dimerization of the monomers, significantly enhancing the extent and rate of dimerization.

While the affinity of the multimer for its target biomolecule(s) often cannot be measured directly due to the dynamic reversible equilibrium with its monomers in an aqueous or biological milieu, it may be possible to extract an apparent multimer-target dissociation constant from a series of experimental determinations. Exploring the effects of a matrix of monomer concentrations, monomer ratios, along with changes in concentration(s) in the target biomolecule(s), coupled with determinations of multimer-monomer dissociation constants, and in some cases additional binding competition, kinetic and biophysical methods, one can extract an estimate of the affinity of the multimeric assembly for its target(s). Through such approaches, one can demonstrate that in some embodiments, the affinity of the multimer for the target biomolecule(s) are less than 1 μM, in some embodiments less than 1 nM, in some embodiments less than 1 pM, in some embodiments less than 1 fM, and in some embodiments less than 1 aM, and in some embodiments less than 1 zM.

Affinities of heterodimerizing monomers for the target biomolecule can be assessed through the testing of the respective monomers in appropriate assays for the target activity or biology because they do not typically self-associate. In contrast, the testing of homodimerizing monomers may not, in some embodiments, afford an affinity for the monomeric or dimeric state, but rather the observed effect (e.g., IC₅₀) is a result of the monomer-dimer dynamics and equilibrium, with the apparent binding affinity (or IC₅₀) being e.g., a weighted measure of the monomer and dimeric inhibitory effects upon the target.

In some cases, the pH of the aqueous fluid in which the multimer forms may be between pH 1 and 9, in some embodiments between pH 1 and 3, in some embodiments between pH 3 and 5, in some embodiments between pH 5 and 7, and in some embodiments between pH 7 and 9. In some embodiments, the multimer may be stable in an aqueous solution having a pH between pH 1 and 9, in some embodiments between pH 1 and 3, in some embodiments between pH 3 and 5, in some embodiments between pH 5 and 7, and in some embodiments between pH 7 and 9. In some embodiments, the aqueous solution may have a physiologically acceptable pH.

In some embodiments, the ligand moiety may be capable of binding to a target and at least partially disrupting a biomolecule-biomolecule interaction (e.g., a protein-protein interaction). In some embodiments, the ligand moiety may be capable of binding to a target and at least partially disrupting a protein-nucleic acid interaction. In some cases, the ligand moiety may be capable of binding to a target and at least partially disrupting a protein-lipid interaction. In some cases, the ligand moiety may be capable of binding to a target and at least partially disrupting a protein-polysaccharide interaction. In some embodiments, the ligand moiety may be capable of at least partially stabilizing a biomolecule-biomolecule interaction. In certain embodiments, the ligand moiety may be capable of at least partially inhibiting a conformational change in a biomolecule target.

In some instances, the linker element may be capable of generating a signal. For example, in some embodiments, the linker element may be capable of fluorescing. In some cases, the linker element may have greater fluorescence when the monomer to which it is attached is part of a multimer as compared to when the monomer to which it is attached is not part of a multimer. In some embodiments, upon multimer formation, the fluorescent brightness of a linker element may increase by at least 2-fold, in some embodiments by at least 5-fold, in some embodiments by at least 10-fold, in some embodiments by at least 50-fold, in some embodiments by at least 100-fold, in some embodiments by at least 1000-fold, and in some embodiments by at least 10000-fold. In some embodiments, a linker element in a multimer may have a peak fluorescence that is red-shifted relative to the peak fluorescence of the linker element in a monomer. In other embodiments, a linker element may have a peak fluorescence that is blue-shifted relative to the peak fluorescence of a linker element in a monomer.

### Monomers

In certain embodiments, a first monomer capable of forming a biologically useful multimer when in contact with one, two or more second monomers in an aqueous media is provided. The first monomer may be represented by the formula:

X¹—Y¹—Z¹ (Formula I)

and pharmaceutically acceptable salts, stereoisomers, metabolites and hydrates thereof, wherein
X¹ is a first ligand moiety capable of binding to and modulating a first target biomolecule;
Y¹ is absent or is a connector moiety covalently bound to X¹ and Z¹;
Z¹ is an activated π-moiety; and

the second monomer has a nucleophile moiety capable of binding with the Z¹ moiety of Formula I to form the multimer.

In some embodiments, Z¹ may be independently selected from the group consisting of:

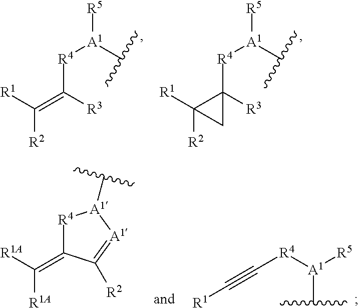

wherein
R¹ and R² are selected, independently for each occurrence, from the group consisting of hydrogen, halo, C₁₋₆alkyl, C₂₋₆alkenyl, C₂₋₆alkynyl, C₃₋₆cycloalkyl, phenyl and heteroaryl; wherein C₁₋₆alkyl, C₂₋₆alkenyl, C₂₋₆alkynyl, C₃₋₆cycloalkyl, phenyl and heteroaryl are optionally substituted with one, two, three or more substituents selected from R^{a};
R^{1A} is selected, independently for each occurrence, from the group consisting of hydrogen, halo, hydroxyl, C₁₋₆alkyl, —O—C₁₋₆alkyl, —NR³R³, C₂₋₆alkenyl, C₂₋₆alkynyl, C₃₋₆cycloalkyl, phenyl and heteroaryl; wherein C₁₋₆alkyl, C₂₋₆alkenyl, C₂₋₆alkynyl, C₃₋₆cycloalkyl, phenyl and heteroaryl are optionally substituted with one, two, three or more substituents selected from R^{a};
R^{a} is independently selected, for each occurrence, from the group consisting of halogen, hydroxyl, C₁₋₆alkyl, C₂₋₆alkenyl, C₃₋₆cycloalkyl, phenyl, heteroaryl, C₁₋₄alkoxy, C(O)C₁₋₆alkyl, C(O)C₁₋₄alkoxy, C(O)NR′R′, sulfonamide, nitro, carboxyl and cyano; wherein C₁₋₆alkyl, C₂₋₆alkenyl, C₃₋₆cycloalkyl, phenyl, heteroaryl, C₁₋₄alkoxy, C(O)C₁₋₆alkyl, C(O)C₁₋₄alkoxy and C(O)NR′R′ are optionally substituted independently, for each occurrence, with one, two, three or more substituents from the group consisting of halogen, hydroxyl, nitro and cyano;
R′ is independently selected, for each occurrence, from the group consisting of H, halogen, hydroxyl, cyano, C₁₋₄alkyl, C₂₋₆alkenyl, C₃₋₆cycloalkyl, phenyl and heteroaryl; wherein C₁₋₄alkyl, C₂₋₆alkenyl, C₃₋₆cycloalkyl, phenyl and heteroaryl are optionally substituted independently, for each occurrence, with one, two, three or more substituents from the group consisting of halogen, hydroxyl, nitro, cyano, C₁₋₄alkyl, C₂₋₆alkenyl and phenyl;
R³ is independently selected, for each occurrence, from the group consisting of hydrogen, and C₁₋₄alkyl; wherein R³ is optionally substituted with one or more substituents selected from R^{a}
R⁴ is independently selected, for each occurrence, from the group consisting of —C(O)—, —C(NR′)—, —SO₂— and —P(O)(OR′)—;
A¹ is independently selected, for each occurrence, from the group consisting of CH, N, and O;
A^{1′} is independently selected, for each occurrence, from the group consisting of CH and N;
R⁵ is independently selected, for each occurrence, from the group consisting of hydrogen and C₁₋₄alkyl; wherein if A¹ is O, there is no R⁵ substitution; or
R¹ and R⁵ may be taken with the atoms to which they are attached to form a 5-7 membered heterocycle; wherein the 5-7 membered heterocycle may optionally have 1 or 2 moieties from the group consisting of oxo, imino and sulfanylidene;
R³ and R⁵ may be taken together with the atoms to which they are attached to form a 4-7 membered heterocycle; wherein the 4-7 membered heterocycle may be substituted by one, two, three or more substituents from the group R^{a}; and wherein two R^{a} substituents may be taken together with the atoms to which they are attached to form a fused aliphatic or heteroaliphatic ring; and

the second monomer has said nucleophile moiety capable of binding with the Z¹ moiety of Formula I to form the multimer.

In some cases, R⁴ may be independently selected, for each occurrence, from the group consisting of —C(O)— and —SO₂—.

In some instances, A¹ may be N.

In some embodiments, R¹ and R² may be hydrogen.

In certain embodiments, Z¹ may be represented by:

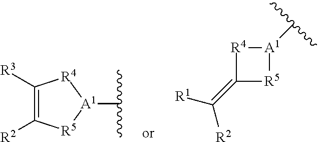

wherein
R¹ and R² are selected, independently for each occurrence, from the group consisting of hydrogen, halogen, C₁₋₆alkyl, C₂₋₆alkenyl and phenyl; wherein C₁₋₆alkyl, C₂₋₆alkenyl and phenyl are optionally substituted with one, two, three or more substituents selected from the group consisting of halogen, hydroxyl and cyano;
R³ is independently selected, for each occurrence, from the group consisting of hydrogen and C₁₋₄alkyl;
R⁴ is independently selected, for each occurrence, from the group consisting of —C(O)— and —SO₂—;
A¹ is N;
R⁵ is —C₁₋₄alkyl-; wherein if A¹ is O, there is no R⁵ substitution; or
R¹ and R⁵ may be taken together to form a 5-7 membered heterocyclic ring; or
R³ and R⁵ may be taken together to form a 5-7 membered heterocyclic ring.

In some instances, Z¹ may be represented by:

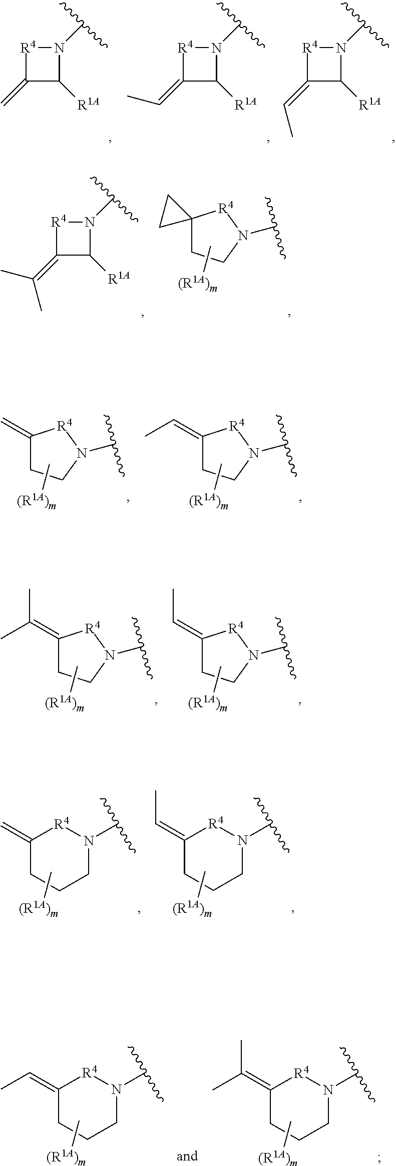

wherein

R⁴ is independently selected, for each occurrence, from the group consisting of —C(O)— and —SO₂—;

m is 0, 1, 2 or more;

R^{1A} is selected, independently for each occurrence, from the group consisting of hydrogen, halo, hydroxyl, C₁₋₆alkyl, —O—C₁₋₆alkyl, —NR^{a}R^{a}, C₂₋₆alkenyl, C₂₋₆alkynyl, C₃₋₆cycloalkyl, phenyl and heteroaryl; wherein C₁₋₆alkyl, C₂₋₆alkenyl, C₂₋₆alkynyl, C₃₋₆cycloalkyl, phenyl and heteroaryl are optionally substituted with one, two, three or more substituents selected from R^{a}; and

R^{a} is independently selected, for each occurrence, from the group consisting of halogen, hydroxyl, C₁₋₆alkyl, C₂₋₆alkenyl, C₃₋₆cycloalkyl, phenyl, heteroaryl, C₁₋₄alkoxy, C(O)C₁₋₆alkyl, C(O)C₁₋₄alkoxy, C(O)NR′R′, sulfonamide, nitro, carboxyl and cyano; wherein C₁₋₆alkyl, C₂₋₆alkenyl, C₃₋₆cycloalkyl, phenyl, heteroaryl, C₁₋₄alkoxy, C(O)C₁₋₆alkyl, C(O)C₁₋₄alkoxy and C(O)NR′R′ are optionally substituted independently, for each occurrence, with one, two, three or more substituents from the group consisting of halogen, hydroxyl, nitro and cyano.

In other embodiments, Z¹ may be represented by:

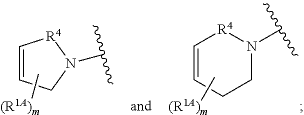

wherein

R⁴ is independently selected, for each occurrence, from the group consisting of —C(O)— and —SO₂—;

m is 0, 1, 2 or more;

R^{1A} is selected, independently for each occurrence, from the group consisting of hydrogen, halo, hydroxyl, C₁₋₆alkyl, —O—C₁₋₆alkyl, —NR^{a}R^{a}, C₂₋₆alkenyl, C₂₋₆alkynyl, C₃₋₆cycloalkyl, phenyl and heteroaryl; wherein C₁₋₆alkyl, C₂₋₆alkenyl, C₂₋₆alkynyl, C₃₋₆cycloalkyl, phenyl and heteroaryl are optionally substituted with one, two, three or more substituents selected from R^{a}; and

R^{a} is independently selected, for each occurrence, from the group consisting of halogen, hydroxyl, C₁₋₆alkyl, C₂₋₆alkenyl, C₃₋₆cycloalkyl, phenyl, heteroaryl, C₁₋₄alkoxy, C(O)C₁₋₆alkyl, C(O)C₁₋₄alkoxy, C(O)NR′R′, sulfonamide, nitro, carboxyl and cyano; wherein C₁₋₆alkyl, C₂₋₆alkenyl, C₃₋₆cycloalkyl, phenyl, heteroaryl, C₁₋₄alkoxy, C(O)C₁₋₆alkyl, C(O)C₁₋₄alkoxy and C(O)NR′R′ are optionally substituted independently, for each occurrence, with one, two, three or more substituents from the group consisting of halogen, hydroxyl, nitro and cyano.

In still other embodiments, Z¹ may be represented by:

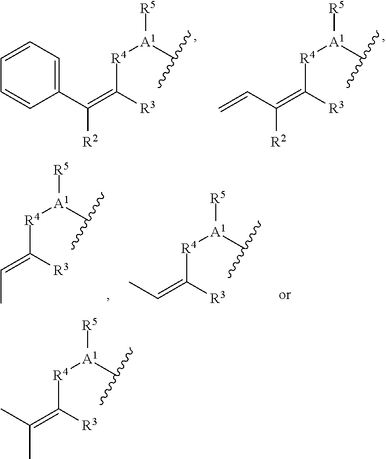

wherein
R² is selected, independently for each occurrence, from the group consisting of hydrogen and C₁₋₆alkyl; wherein C₁₋₆alkyl is optionally substituted with one, two, three or more substituents selected from the group consisting of halogen, hydroxyl and cyano;
R³ is independently selected, for each occurrence, from the group consisting of hydrogen and C₁₋₄alkyl;
R⁴ is independently selected, for each occurrence, from the group consisting of —C(O)— and —SO₂—;
A¹ is N;
R⁵ is independently selected, for each occurrence, from the group consisting of hydrogen and C₁₋₄alkyl; wherein R³ and R⁵ may be taken together to form a 5-7 membered heterocyclic ring.

In certain embodiments, Z¹ may be represented by:

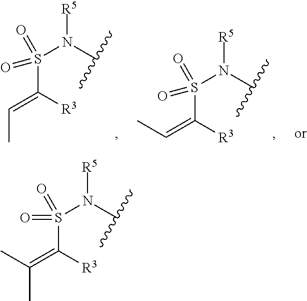

wherein
R³ is independently selected, for each occurrence, from the group consisting of hydrogen and C₁₋₄alkyl;
R⁵ is independently selected, for each occurrence, from the group consisting of hydrogen and C₁₋₄alkyl; wherein R³ and R⁵ may be taken together to form a 5-7 membered heterocyclic ring.

In some cases, Z¹ may be represented by:

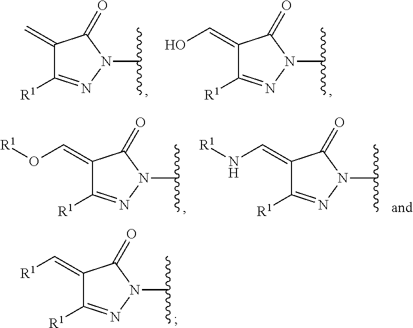

R¹ is selected, independently for each occurrence, from the group consisting of hydrogen, halo, C₁₋₆alkyl, C₂₋₆alkenyl, C₂₋₆alkynyl, C₃₋₆cycloalkyl, phenyl and heteroaryl; wherein C₁₋₆alkyl, C₂₋₆alkenyl, C₂₋₆alkynyl, C₃₋₆cycloalkyl, phenyl and heteroaryl are optionally substituted with one, two, three or more substituents selected from R^{a};

R^{a} is independently selected, for each occurrence, from the group consisting of halogen, hydroxyl, C₁₋₆alkyl, C₂₋₆alkenyl, C₃₋₆cycloalkyl, phenyl, heteroaryl, C₁₋₄alkoxy, C(O)C₁₋₆alkyl, C(O)C₁₋₄alkoxy, C(O)NR′R′, sulfonamide, nitro, carboxyl and cyano; wherein C₁₋₆alkyl, C₂₋₆ alkenyl, C₃₋₆cycloalkyl, phenyl, heteroaryl, C₁₋₄alkoxy, C(O)C₁₋₆alkyl, C(O)C₁₋₄alkoxy and C(O)NR′R′ are optionally substituted independently, for each occurrence, with one, two, three or more substituents from the group consisting of halogen, hydroxyl, nitro and cyano;

In some embodiments, the first monomer may form a biologically useful multimer when in contact with one, two, three or more second monomers in vivo. In some cases, the first monomer may form a biologically useful dimer when in contact with a second monomer in vivo. In some instances, the first monomer may form a biologically useful trimer when in contact with two second monomers.

In certain embodiments, X¹ may be a non-peptidyl ligand moiety. In some embodiments, the ligand moiety is a pharmacophore.

In some cases, the second monomer may be represented by:

X²—Y²—Z² (Formula II),

and pharmaceutically acceptable salts, stereoisomers, metabolites and hydrates thereof, wherein
X² is a second ligand moiety capable of binding to and modulating a second target biomolecule;
Y² is absent or is a connector moiety covalently bound to X² and Z²;
Z² is said nucleophile moiety.

In some instances, X¹ and X² may be the same. In some embodiments, X¹ and X² may be different.

In some embodiments, the first target biomolecule and the second target biomolecule may be substantially different. In some cases, the first target biomolecule and the second target biomolecule may be the same. In certain embodiments, the target biomolecule may be a protein target. In some instances, the first target biomolecule may be a ribosome. In other instances, the first target biomolecule may be a tryptase.

In some embodiments, the IC₅₀ of the first monomer against a first target biomolecule and the IC₅₀ of the second monomer against a second target biomolecule may be greater than the apparent IC₅₀ of a combination of the monomers against the first target biomolecule and the second target biomolecule. The combination of monomers may be any suitable ratio. For example, the ratio of the first monomer to the second monomer may be between 10:1 to 1:10, in some embodiments between 5:1 and 1:5, and in some embodiments between 2:1 and 1:2. In some cases, the ratio of the first monomer to the second monomer may be essentially 1:1. In some instances, the ratio of the smaller of the IC₅₀ of the first monomer and the second monomer to the apparent IC₅₀ of the multimer may be at least 3.0. In other instances, the ratio of the smaller IC₅₀ of the first monomer or the second monomer to the apparent IC₅₀ of the multimer may be at least 10.0. In some embodiments, the ratio of the smaller IC₅₀ of the first monomer or the second monomer to the apparent IC₅₀ of the multimer may be at least 30.0.

For example, for disclosed monomers forming a heteromultimer, the apparent IC₅₀ resulting from an essentially equimolar combination of monomers against the first target biomolecule and the second target biomolecule is at least about 3 to 10 fold lower, at least about 10 to 30 fold lower, at least about 30 fold lower, or at least about 40 to 50 fold lower than the lowest of the IC₅₀ of the second monomer against the second target biomolecule or the IC₅₀ of the first monomer against the first target biomolecule.

In some embodiments, the aqueous media may have a physiologically acceptable pH.

In certain embodiments, Z² may be independently selected, for each occurrence, from the group consisting of:

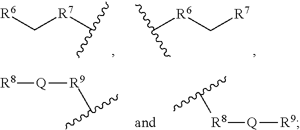

wherein
R⁶ is independently selected, for each occurrence, from the group consisting of hydrogen, C₁₋₄alkyl, phenyl, heteroaryl, C(O)NR^{b}R^{b}, and R⁷; wherein C₁₋₄alkyl, phenyl and heteroaryl are optionally substituted independently, for each occurrence, with R^{b}.
R^{b} is independently selected, for each occurrence, from the group consisting of H, halogen, hydroxyl, cyano, C₁₋₄alkyl, C₂₋₆alkenyl, C₃₋₆cycloalkyl, phenyl and heteroaryl; wherein C₁₋₄alkyl, C₂₋₆alkenyl, C₃₋₆cycloalkyl, phenyl and heteroaryl are optionally substituted independently, for each occurrence, with one, two, three or more substituents from the group consisting of halogen, hydroxyl, nitro, cyano, C₁₋₄alkyl, C₂₋₆alkenyl and phenyl;
R⁷ is independently selected, for each occurrence, from the group consisting of C(O)—C₁₋₄alkyl, C(O)-phenyl, C(O)-heteroaryl, CO₂— C₁₋₄alkyl, CO₂-phenyl, CHO, cyano and nitro;
R⁸ and R⁹ independently selected, for each occurrence, from the group consisting of hydrogen, C₁₋₄alkyl, phenyl, and heteroaryl; wherein C₁₋₄alkyl, phenyl and heteroaryl are optionally substituted independently, for each occurrence, with R_{b};
Q is independently selected, for each occurrence, from the group consisting of —O—, —S—, and —NR^{b}—.

In some cases, the first monomer may form a biologically useful multimer when in contact with one, two, three or more second monomers in vivo. For example, the multimer may be a biologically useful dimer when the first monomer is in contact with the second monomer. Alternatively, the multimer may be a biologically useful trimer when the first monomer is in contact with two second monomers. In other instances, the multimer may be a biologically useful cyclic tetramer when the first monomer is in contact with three second monomers.

As discussed above, the ligand moiety may be a pharmacophore and the target biomolecule may be a protein target. In some cases, the first target biomolecule and the second target biomolecule may be the same. In other cases, the first target biomolecule and the second target biomolecule may be different. For example, the first target biomolecule may be a ribosome. In another embodiment, the first target biomolecule may be a tryptase. Alternatively, the second target biomolecule may be a ribosome. In another embodiment, the second target biomolecule may be a tryptase.

In other cases, X¹ may be a non-peptidyl ligand moiety. In one embodiment, X¹ and X² may be the same. In another embodiment, X¹ and X² may be the different.

In some embodiments, the effects of the multimer formed from the monomers may be greater than the sum of the effects of the individual monomers. For example, the ratio of the smaller of the apparent IC₅₀ of the first monomer and the second monomer to the apparent IC₅₀ of the multimer may be at least 3.0, 10.0 or 30.0.

In certain embodiments, the first monomer and the second monomer may reversibly associate to form the multimer.

As discussed above, a monomer may be capable of reacting with one or more other monomers to form a multimer in an aqueous composition, e.g. in vivo. In some embodiments, a first monomer may react with a second monomer to form a dimer. In other embodiments, a first monomer may react with two second monomers to form a trimer. In still other embodiments, a first monomer may react with three second monomers to form a cyclic tetramer. In some embodiments, each of the monomers that form a multimer may be essentially the same. In some embodiments, each of the monomers that form a multimer may be substantially different. In certain embodiments, at least some of the monomers that form a multimer may be essentially the same or may be substantially different.

In some embodiments, the linker element of a first monomer and the linker element of a second monomer may be substantially different. In other embodiments, the connector element of a first monomer and the connector element of a second monomer may be substantially different. In still other embodiments, the ligand moiety (e.g., pharmacophore) of a first monomer and the ligand moiety (e.g. pharmacophore) of the second monomer may be substantially different.

In some cases, formation of a multimer from a plurality of monomers may be irreversible. In some embodiments, formation of a multimer from a plurality of monomers may be reversible. For example, in some embodiments, the multimer may have an oligomer or dimer dissociation constant between 10 mM and 1 nM, in some embodiments between 1 mM and 100 nM, in some embodiments between 1 mM and 1 mM, and in some embodiments between 500 mM and 1 mM. In certain embodiments, the multimer may have a dissociation constant of less than 10 mM, in some embodiments less than 1 mM, in some embodiments less than 500 mM, in some embodiments less than 100 mM, in some embodiments less than 50 mM, in some embodiments less than 1 mM, in some embodiments less than 100 nM, and in some embodiments less than 1 nM.

### Multimers

In some embodiments, a first monomer and a second monomer may form a dimer in aqueous solution. For example, in some instances, the first monomer may form a biologically useful dimer with a second monomer in vivo.

In some embodiments, a therapeutic multimer compound formed from the multimerization in an aqueous media of a first monomer may be represented by:

X¹—Y¹—Z¹ (Formula I)

and pharmaceutically acceptable salts, stereoisomers, metabolites and hydrates thereof,

and a second monomer represented by:

X²—Y²—Z² (Formula II)

and pharmaceutically acceptable salts, stereoisomers, metabolites and hydrates thereof

In some cases, X¹ may be a first ligand moiety capable of binding to and modulating a first target biomolecule;

Y¹ is absent or is a connector moiety covalently bound to X¹ and Z¹;

Z¹ is selected from the group consisting of:

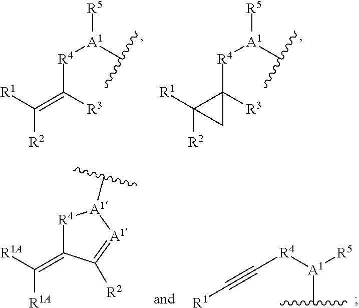

wherein
R¹ and R² are selected, independently for each occurrence, from the group consisting of hydrogen, halo, C₁₋₆alkyl, C₂₋₆alkenyl, C₂₋₆alkynyl, C₃₋₆cycloalkyl, phenyl and heteroaryl; wherein C₁₋₆alkyl, C₂₋₆alkenyl, C₂₋₆alkynyl, C₃₋₆cycloalkyl, phenyl and heteroaryl are optionally substituted with one, two, three or more substituents selected from R^{a};
R^{1A} is selected, independently for each occurrence, from the group consisting of hydrogen, halo, hydroxyl, C₁₋₆alkyl, —O—C₁₋₆alkyl, —NR³R³, C₂₋₆alkenyl, C₂₋₆alkynyl, C₃₋6cycloalkyl, phenyl and heteroaryl; wherein C₁₋₆alkyl, C₂₋₆alkenyl, C₂₋₆alkynyl, C₃₋₆cycloalkyl, phenyl and heteroaryl are optionally substituted with one, two, three or more substituents selected from R^{a};
R^{a} is independently selected, for each occurrence, from the group consisting of halogen, hydroxyl, C₁₋₆alkyl, C₂₋₆alkenyl, C₃₋₆cycloalkyl, phenyl, heteroaryl, C₁₋₄alkoxy, C(O)C₁₋₆alkyl, C(O)C₁₋₄alkoxy, C(O)NR′R′, sulfonamide, nitro, carboxyl and cyano; wherein C₁₋₆alkyl, C₂₋₆alkenyl, C₃₋₆cycloalkyl, phenyl, heteroaryl, C₁₋₄alkoxy, C(O)C₁₋₆alkyl, C(O)C₁₋₄alkoxy and C(O)NR′R′ are optionally substituted independently, for each occurrence, with one, two, three or more substituents from the group consisting of halogen, hydroxyl, nitro and cyano;
R′ is independently selected, for each occurrence, from the group consisting of H, halogen, hydroxyl, cyano, C₁₋₄alkyl, C₂₋₆alkenyl, C₃₋₆cycloalkyl, phenyl and heteroaryl; wherein C₁₋₄alkyl, C₂₋₆alkenyl, C₃₋₆cycloalkyl, phenyl and heteroaryl are optionally substituted independently, for each occurrence, with one, two, three or more substituents from the group consisting of halogen, hydroxyl, nitro, cyano, C₁₋₄alkyl, C₂₋₆alkenyl and phenyl;
R³ is independently selected, for each occurrence, from the group consisting of hydrogen, and C₁₋₄alkyl; wherein R³ is optionally substituted with one or more substituents selected from R^{a}
R⁴ is independently selected, for each occurrence, from the group consisting of —C(O)—, —C(NR′)—, —SO₂— and —P(O)(OR′)—;
A¹ is independently selected, for each occurrence, from the group consisting of CH, N, and O;
A^{1′} is independently selected, for each occurrence, from the group consisting of CH and N;
R⁵ is independently selected, for each occurrence, from the group consisting of hydrogen and C₁₋₄alkyl; wherein if A¹ is O, there is no R⁵ substitution; or
R¹ and R⁵ may be taken with the atoms to which they are attached to form a 5-7 membered heterocycle; wherein the 5-7 membered heterocycle may optionally have 1 or 2 moieties from the group consisting of oxo, imino and sulfanylidene;
R³ and R⁵ may be taken together with the atoms to which they are attached to form a 4-7 membered heterocycle; wherein the 4-7 membered heterocycle may be substituted by one, two, three or more substituents from the group R^{a}; and wherein two R^{a} substituents may be taken together with the atoms to which they are attached to form a fused aliphatic or heteroaliphatic ring; and
X² is a second ligand moiety capable of binding to and modulating a second target biomolecule;
Y² is absent or is a connector moiety covalently bound to X² and Z²;
Z² is selected from the group consisting of:

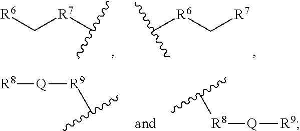

wherein
R⁶ is independently selected, for each occurrence, from the group consisting of hydrogen, C₁₋₄alkyl, phenyl, heteroaryl, C(O)NR^{b}R^{b}, and R⁷; wherein C₁₋₄alkyl, phenyl and heteroaryl are optionally substituted independently, for each occurrence, with R_{b};
R^{b} is independently selected, for each occurrence, from the group consisting of H, halogen, hydroxyl, cyano, C₁₋₄alkyl, C₂₋₆alkenyl, C₃₋₆cycloalkyl, phenyl and heteroaryl; wherein C₁₋₄alkyl, C₂₋₆alkenyl, C₃₋₆cycloalkyl, phenyl and heteroaryl are optionally substituted independently, for each occurrence, with one, two, three or more substituents from the group consisting of halogen, hydroxyl, nitro, cyano, C₁₋₄alkyl, C₂₋₆alkenyl and phenyl;
R⁷ is independently selected, for each occurrence, from the group consisting of C(O)—C₁₋₄alkyl, C(O)-phenyl, C(O)-heteroaryl, CO₂— C₁₋₄alkyl, CO₂-phenyl, CHO, cyano and nitro;
R⁸ and R⁹ independently selected, for each occurrence, from the group consisting of hydrogen, C₁₋₄alkyl, phenyl, and heteroaryl; wherein C₁₋₄alkyl, phenyl and heteroaryl are optionally substituted independently, for each occurrence, with R_{b}.
Q is independently selected, for each occurrence, from the group consisting of —O—, —S—, and —NR^{b}—.

In some embodiments, the formation of the multimer may be substantially irreversible in an aqueous media. In some instances, formation of the multimer may be substantially reversible in an aqueous media.

In some cases, X¹ and X² may be the same. In other cases, X¹ and X² may be different.

### Connectors

In some embodiments, a monomer may comprise a connector that joins the ligand moiety with the linker element. In some instances, such connectors do not have significant binding or other affinity to an intended target. However, in certain embodiments, a connector may contribute to the affinity of a ligand moiety to a target.

In some embodiments, a connector element may be used to connect the linker element to the ligand moiety. In some instances, the connector element may be used to adjust spacing between the linker element and the ligand moiety. In some cases, the connector element may be used to adjust the orientation of the linker element and the ligand moiety. In certain embodiments, the spacing and/or orientation the linker element relative to the ligand moiety can affect the binding affinity of the ligand moiety (e.g., a pharmacophore) to a target. In some cases, connectors with restricted degrees of freedom are preferred to reduce the entropic losses incurred upon the binding of a multimer to its target biomolecule. In some embodiments, connectors with restricted degrees of freedom are preferred to promote cellular permeability of the monomer.

In some embodiments, the connector element may be used for modular assembly of monomers. For example, in some instances, a connector element may comprise a functional group formed from reaction of a first and second molecule. In some cases, a series of ligand moieties may be provided, where each ligand moiety comprises a common functional group that can participate in a reaction with a compatible functional group on a linker element. In some embodiments, the connector element may comprise a spacer having a first functional group that forms a bond with a ligand moiety and a second functional group that forms a bond with a linker element.

Contemplated connectors may be any acceptable (e.g. pharmaceutically and/or chemically acceptable) bivalent linker that, for example, does not interfere with multimerization of the disclosed monomers. For instance, such linkers may be substituted or unsubstituted C₁-C₁₀ alkylene, substituted or unsubstituted cycloalkylene, substituted or unsubstituted phenyl or naphthyl, substituted or unsubstituted heteroaryl, acyl, sulfone, phosphate, ester, carbamate, or amide. Contemplated connectors may include polymeric connectors, such a polyethylene glycol (e.g.,

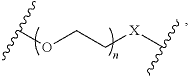

where n is 1, 2, 3, 4, 5, 6, 7, 8, 9, 10, 11, 12, 13, 14, 15, 16, 17, 18, 19, or 20, and X is C; O; S(O)_{q}, where q is 0, 1, or 2; NH; N-alkyl; or —C(O)—) or other pharmaceutically acceptable polymers. For example, contemplated connectors may be a covalent bond or a bivalent C₁₋₂₀ saturated or unsaturated, straight or branched, hydrocarbon chain, wherein one, two, or three or four methylene units of the hydrocarbon chain are optionally and independently replaced by cyclopropylene, —NR—, —N(R)C(O)—, —C(O)N(R)—, —N(R)SO₂—, —SO₂N(R)—, —O—, —C(O)—, —OC(O)—, —C(O)O—, —S—, —SO—, —SO₂—, —C(═NR)—, phenyl, naphthyl, or a mono or bicyclic heterocycle ring, where R is H or C₁₋₆alkyl. In some embodiments, a connector may be from about 7 atoms to about 13 atoms in length, or about 8 atoms to about 12 atoms, or about 9 atoms to about 11 atoms in length. For purposes of counting connector length when a ring is present in the connector group, the ring is counted as three atoms from one end to the other.

In some embodiments, a connector may have the following structure:

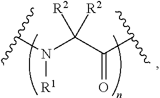

where:

n is 1, 2, 3, 4, 5, 6, 7, 8, 9, 10, 11, 12, 13, 14, 15, 16, 17, 18, 19, or 20;

R¹ and R² are, independently for each occurrence, selected from the group consisting of H, C₁₋₆alkyl, C₁₋₆heteroalkyl, phenyl, or heteroaryl, wherein alkyl, heteroalkyl, phenyl, and heteroaryl are optionally substituted with —OH, —NH₂, —SH, —COOH, —C(O)NH₂, halo, phenyl, and heteroaryl; or

R¹ and R², or R² and R², together with the atoms to which they are attached, form a heterocyclic structure optionally substituted with —OH, —NH₂, —SH, —COOH, —C(O)NH₂, halo, phenyl, and heteroaryl.

In some embodiments, a connector may comprise a phenyl, naphthyl, or mono or bicyclic heteroaryl ring, each optionally substituted. For example, a connector may comprise one or more of the following aryl structures:

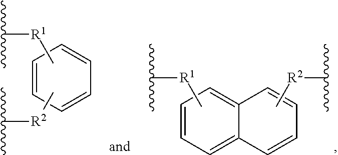

where R¹ and R² are the remainder of the connector. A person of skill in the art would recognize that some substitutions may be chemically less stable and hence less preferred.

In another embodiment, a connector may comprise a triazole ring having the following structure:

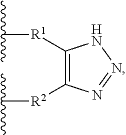

where R¹ and R² are the remainder of the connector. For example, a monomer comprising a triazole-containing connector may have the following general structure:

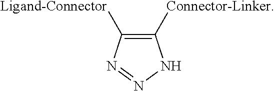

Such triazole-joined compounds may be formed, e.g., as a result of a “click” type reaction (i.e., an azide-alkyne cycloaddition). For example, a first segment of a connector having a terminal alkyne and a second segment of a connector having a terminal azide may be joined by a “click” reaction to form a single connector joined by a triazole, as shown above. In some embodiments, the first connector and the second connector each are less than or equal to 20 atoms in length, or in some embodiments each are less than or equal to 12 atoms in length.

In another embodiment, a connector moiety may maximally span from about 5 Å to about 50 Å, in some embodiments about 5 Å to about 25 Å, in some embodiments about 20 Å to about 50 Å, in some embodiments about 20 Å to about 30 Å, and in some embodiments about 6 Å to about 15 Å in length. For purposes of counting connector length when a ring is present in the connector group, the ring is counted as three atoms from one end to the other. In another embodiment, a connector moiety may maximally span from about 1 Å to about 20 Å, in some embodiments about 1 Å to about 10 Å, in some embodiments about 1 Å to about 5 Å and in some embodiments about 5 Å to about 15 Å in length. For example, a connector moiety may maximally span about 1 Å, about 3 Å, about 5 Å, about 7 Å, about 9 Å, about 11 Å, about 13 Å, about 15 Å, about 17 Å, or about 19 Å.

In some embodiments, a connector may be selected from the group consisting of:

—NR¹³—(CH₂—CH₂—O)ₛ—CH₂—CH₂—NR¹³—C(O)—; —(O—CH₂—CH₂)ₜ—NR¹³—C(O)—; —O—C₅₋₁₀alkyl-NR¹³—C(O)—; -heterocyclyl-C(O)—; —N(C₁₋₃alkyl)-C₁₋₆alkyl-NH—C(O)—; —NH—C₁₋₆alkyl-N(C₁₋₃ alkyl)-C(O)—; —NR¹³—C₆₋₁₅alkyl-NR¹³—C(O)—; -heterocyclyl-C₀₋₆alkyl-NR¹³—C(O)—; and —NR¹³—C₀₋₆alkyl-heterocyclyl-C(O)—;

wherein, independently for each occurrence,
R¹³ is selected from the group consisting of H and C₁₋₆alkyl;
s is an integer from 0-10 (i.e., 0, 1, 2, 3, 4, 5, 6, 7, 8, 9, or 10); and
t is an integer from 0-10 (i.e., 0, 1, 2, 3, 4, 5, 6, 7, 8, 9, or 10).

In certain embodiments, heterocyclyl may be a 5-7 membered heterocyclic ring comprising 1 or 2 nitrogen atoms.

In certain embodiments, R¹³ may be H. In certain other embodiments, R¹³ may be C₁₋₆alkyl. For example, in some embodiments, R¹³ may be methyl.

For example, in some embodiments, a connector may be selected from the group consisting of:

—NH—(CH₂—CH₂—O)ₛ—CH₂—CH₂—NH—C(O)—; —(O—CH₂—CH₂)ₜ—NH—C(O)—; —O—(CH₂)ₜ—NH—C(O)—; —N(CH₃)—(CH₂)₂—NH—C(O)—; —NH—(CH₂)₂—N(CH₃)—C(O); —NH—(CH₂)ᵤ—NH—C(O)—; —O—CH₂—C(O)—;

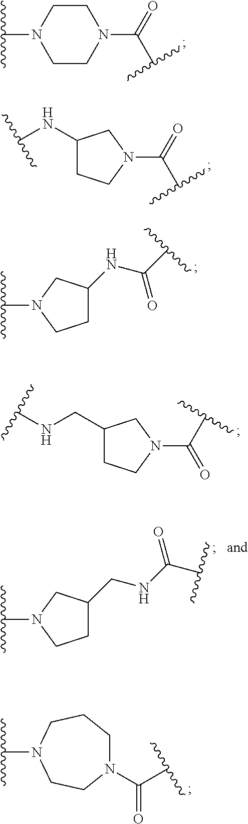

wherein u is an integer from 2-15 (i.e., 2, 3, 4, 5, 6, 7, 8, 9, 10, 11, 12, 13, 14, or 15).

In certain embodiments, a connector may be selected from the group consisting of:

—NR¹³—C₆₋₁₅alkyl-NR¹³—C(O)—; —NR¹³—(CH₂—CH₂—O)ₛ—C₁₋₆alkyl-NR¹³—C(O)—; —(O—CH₂—CH₂)ₛ—NR¹³—C(O)—; —S—C₀₋₆alkyl-; —NR¹³—C₃₋₆alkyl-; —SO₂—NR¹³—C₀₋₆alkyl-; —SO₂-heterocyclyl-C₀₋₆alkyl-; -heterocyclyl-C(O)—; -heterocyclyl-C₀₋₆alkyl-NR¹³—C(O)—; —NR¹³—C₀₋₆alkyl-heterocyclyl-C(O)—; —O—C₁₋₆alkyl-C(O)—; —O—C₁₋₁₅alkyl-NR¹³—C(O)—; —O—C₁₋₁₅alkyl-C(O)—NR¹³—; and —O—C₁₋₆ alkyl-, wherein C₁₋₆alkyl is optionally substituted by —OH;

wherein, independently for each occurrence,

R¹³ is selected from the group consisting of H and C₁₋₆alkyl; and

s is an integer from 1-15.

In certain embodiments, heterocyclyl may be a 5-7 membered heterocyclic ring comprising 1 or 2 nitrogen atoms.

In certain embodiments, R¹³ may be H. In certain other embodiments, R¹³ may be C₁₋₆alkyl. For example, in some embodiments, R¹³ may be methyl.

In certain embodiments, a connector may be selected from the group consisting of:

—NH—(CH₂—CH₂—O)ₛ—CH₂—CH₂—NH—C(O)—; —(O—CH₂—CH₂)ₛ—NH—C(O)—; —S—; —S—CH₂—; —O—(CH₂)ₛ—NH—C(O)—; —SO₂—NH—; —SO₂—NH—CH₂—; —N(CH₃)—(CH₂)₂—NH—C(O)—; —NH—(CH₂)₂—N(CH₃)—C(O); —NH—(CH₂)ᵤ—NH—C(O)—; —O—CH₂—C(O)—;

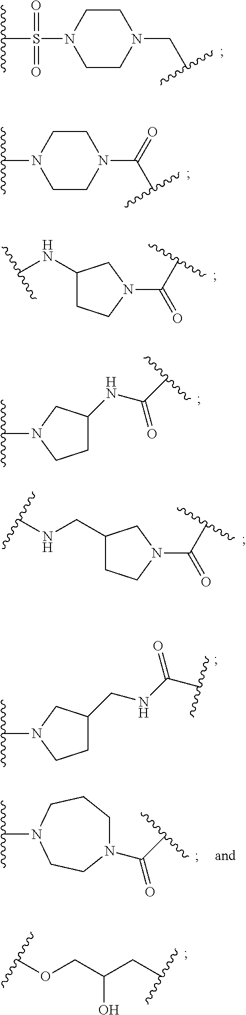

wherein u is an integer from 6-15.

In some embodiments, a connector may be selected from the group consisting of:

—NR¹³—(CH₂—CH₂—O)ₛ—C₁₋₆alkyl-NR¹³—C(O)—; —(O—CH₂—CH₂)ₛ—NR¹³—C(O)—; —S—C₀₋₆alkyl-; —NR¹³—C₀₋₆alkyl-; —SO₂—NR¹³—C₀₋₆alkyl-; —SO₂-heterocyclyl-C₀₋₆alkyl-; -heterocyclyl-C(O)—; -heterocyclyl-C₀₋₆alkyl-NR¹³—C(O)—; —NR¹³—C₀₋₆alkyl-heterocyclyl-C(O)—; —O—C₁₋₆alkyl-C(O)—; —O—C₁₋₁₅alkyl-NR¹³—C(O)—; and —O—C₁₋₆alkyl-, wherein C₁₋₆alkyl is optionally substituted by —OH; wherein, independently for each occurrence, s is an integer from 0-10 and R¹³ is selected from the group consisting of H and C₁₋₆alkyl.

### Methods

In some embodiments, a method of administering a pharmaceutically effective amount of a multimeric compound to a patient in need thereof is provided. In some cases, the method comprises administering to the patient thereof an amount of the first monomer and an amount of the second monomer in amounts effective such that the pharmaceutically effective amount of the resulting multimer is formed in vivo. For example, the multimer may be a dimer. Alternatively, the multimer may be a trimer.

In some embodiments, a first monomer and a second monomer may be administered substantially sequentially. In other embodiments, the first monomer and the second monomer are administered substantially simultaneously. In some embodiments the monomers may be administered, sequentially or simultaneously, by different routes of administration. In still further embodiments, a first monomer and a second monomer may be administered after forming a multimer.

In some embodiments, two or more target biomolecules may be modulated substantially simultaneously. For example, an aqueous composition comprising a bimolecular target may be contacted with a first monomer represented by:

X¹—Y¹—Z¹ (Formula I)
and pharmaceutically acceptable salts, stereoisomers, metabolites and hydrates thereof, wherein
X¹ is a first ligand moiety capable of binding to and modulating a first target biomolecule; and
   a second monomer represented by:

X²—Y²—Z² (Formula II),
and pharmaceutically acceptable salts, stereoisomers, metabolites and hydrates thereof, wherein
X₂ is a ligand moiety capable of binding to and modulating a second target biomolecule;

wherein upon contact with the aqueous composition, said first monomer and said second monomer forms a multimer that binds to the first target biomolecule and the second target biomolecule.

In some cases, a disease associated with two or more target biomolecules in a patient in need thereof may be treated. For example, a first monomer may be administered to a patient, where the first monomer may represented by:

X¹—Y¹—Z¹ (Formula I)

and pharmaceutically acceptable salts, stereoisomers, metabolites and hydrates thereof, wherein

X¹ is a first ligand moiety capable of binding to and modulating a first target biomolecule; and

administering to said patient a second monomer represented by:

X²—Y²—Z² (Formula II), wherein

X² is a second ligand moiety capable of binding to and modulating a second target biomolecule, wherein upon administration, said first monomer and said second monomer forms a multimer in vivo that binds to the first target biomolecule and the second target biomolecule. In some embodiments, the disease may be chronic obstructive pulmonary disease (COPD).

In some embodiments, the target biomolecule may be a protein. In some instances, the target biomolecule may be a protein domain.

In some instances, the first monomer and the second monomer may be administered substantially sequentially. In other instances, the first monomer and the second monomer may be administered substantially simultaneously.

In some embodiments, a patient in need thereof may have chronic obstructive pulmonary disease (COPD).

In some embodiments, the target biomolecule may be a protein. Alternatively, the target biomolecule may be a protein domain. In other embodiments, the target biomolecule may be nucleic acid. In some cases, the ligand moiety may be a pharmacophore.

In some embodiments, a multimer may be used to inhibit or facilitate protein-protein interactions. For example, in some cases, a multimer may be capable of activating or inactivating a signaling pathway. Without wishing to be bound by any theory, a multimer may bind to a target protein and affect the conformation of the target protein such that the target protein is more biologically active as compared to when the multimer does not bind the target protein. In some embodiments monomers may be chosen such that a multimer formed from the monomers binds to at least two regions of a target molecule.

Without wishing to be bound by any theory, protein-protein and protein-nucleic acid recognition often work through protein interaction domains, such as the SH2, SH3, and PDZ domains. Currently, there are over 75 such motifs reported in the literature (Hunter, et al., Cell 100:113-127 (2000); Pawson et al., Genes & Development 14:1027-1047 (2000)). For example, SH2 domains are miniature receptors for protein regions containing a phosphorylated tyrosine. SH2 domains may be found in proteins that act as, or play a role in, for example, adaptors, scaffolds, kinases, phosphatases, ras signalling, transcription, ubiquitination, cytoskeletal regulation, signal regulation, and phospholipid second messenger signaling. As another non-limiting example, SH3 domains bind peptide loops with the motif RXXK or PXXP. Many proteins have both SH2 and SH3 domains, which act as “receptors” to bind one or more protein partners. Coferons may be designed to inhibit binding of a phosphotyrosine protein to its cognate SH2 domain. Alternatively, monomers and multimers may be designed so one ligand binds one motif (i.e. SH2), and a second ligand binds a second motif (i.e. SH3), either on the same or different proteins.

Many large proteins or macromolecular complexes (e.g., ribosomes) have multiple binding sites with known drug inhibitors. In some embodiments, linker elements may be used to bring together two pharmacophores on the same target to: (i) bind the target with higher affinity; (ii) exhibit a stronger inhibition than either pharmacophore alone; (iii) exhibit greater activation than either pharmacophore alone; or (iv) create a binding entity covering a larger surface area of the target, making it harder for the organism/cell/virus to develop resistance to the drug via point mutations.

In some embodiments, a multimer may target a tryptase. For example, a multimer may be used to treat conditions activated by a trypase, such as mast cell mediated inflammatory conditions (e.g. asthma). Asthma is frequently characterized by progressive development of hyper-responsiveness of the trachea and bronchi to both immunospecific allergens and generalized chemical or physical stimuli, which lead to the onset of chronic inflammation. Leukocytes containing IgE receptors, notably mast cells and basophils, are present in the epithelium and underlying smooth muscle tissues of bronchi. These leukocytes initially become activated by the binding of specific inhaled antigens to the IgE receptors and then release a number of chemical mediators. For example, degranulation of mast cells leads to the release of proteoglycans, peroxidase, arylsulfatase B, chymase, and tryptase, which results in bronchiole constriction.

Human mast cell β-tryptase-II is a tetrameric serine protease that is concentrated in mast cell secretory granules. The enzyme is involved in IgE-induced mast cell degranulation in an allergic response and is potentially a target for the treatment of allergic asthma, rhinitis, conjunctivitis and dermatitis. Tryptase has also been implicated in the progression of renal, pulmonary, hepatic, testicular fibrosis, chronic obstructive pulmonary disease (COPD) and inflammatory conditions such as ulcerative colitis, inflammatory bowel disease, rheumatoid arthritis, and various other mast cell-related diseases. In some embodiments, multimers may be used to treat such diseases.

Tryptase is stored in the mast cell secretory granules and is the major protease of human mast cells. Tryptase has been implicated in a variety of biological processes, including degradation of vasodilatory and bronchodilatory neuropeptides and modulation of bronchial responsiveness to histamine. As a result, tryptase inhibitors may be useful as anti-inflammatory agents for treatment of inflammatory disease and may also be useful in treating or preventing allergic rhinitis, inflammatory bowel disease, psoriasis, ocular or vernal or ulcerative conjunctivitis, dermatological conditions (e.g., psoriasis, eczema, or atopic dermatitis), arthritis (e.g., rheumatoid arthritis, osteoarthritis, hematoid arthritis, traumatic arthritis, rubella arthritis, psoriatic arthritis, or gouty arthritis), rheumatoid spondylitis, interstitial lung disease, chronic obstructive pulmonary disease, and diseases of joint cartilage destruction.

In addition, tryptase has been shown to be a potent mitogen for fibroblasts, suggesting its involvement in the pulmonary fibrosis in asthma and interstitial lung diseases. Therefore, in some embodiments, tryptase inhibitors may be useful in treating or preventing fibrotic conditions, for example, fibrosis, sceleroderma, pulmonary fibrosis, liver cirrhosis, myocardial fibrosis, neurofibromas, hepatic fibrosis, renal fibrosis, testicular, and hypertrophic scars.

Additionally, tryptase inhibitors may be useful in treating or preventing myocardial infarction, stroke, angina and other consequences of atherosclerotic plaque rupture.

Tryptase has also been discovered to activate prostromelysin that in turn activates collagenase, thereby initiating the destruction of cartilage and periodontal connective tissue, respectively. In some embodiments, tryptase inhibitors may be useful in the treatment or prevention of arthritis, periodontal disease, diabetic retinopathy, a condition relating to atherosclerotic plaque rupture, anaphylatis ulcerative colitis, and tumour growth. Also, tryptase inhibitors may be useful in the treatment of anaphylaxis, multiple sclerosis, peptic ulcers, and syncytial viral infections.

A variety of antibiotics elicit their antibacterial activity by binding to the bacterial ribosome and inhibiting protein synthesis. Many of these antibiotics bind the peptidyl transferase center of the ribosome (P site). In some embodiments, a multimer may bind to two or more sites on the ribosome. For example, a first pharmacophore of a multimer may bind to the peptidyl transferase center of the ribosome (i.e., the P site) and a second multimer may bind to site adjacent to the P site. As a non-limiting, illustrative example, Linezolid, an oxazolidinone antibiotic, is believed to bind adjacent to the binding site for Sparsomycin. The close juxtaposition of the linezolid binding site with the sparosmycin binding site presents a possible scenario for developing monomers based on linezolid and sparsomycin that can dimerize on binding to the ribosome, thereby creating a high affinity and high specificity inhibitor of bacterial protein synthesis.

Other non-limiting examples of target protein families are provided in Table 1 below. Also provided in Table 1 are endogenous ligands, agonists, and antagonists that bind to the protein families. Examples of detection assays are also provided in Table 1, which may be used in a screening assay to detect activation and/or inhibition of the target protein.

Provided in Table 2 are non-limiting examples of domains that can bind a ligand, proteins that contain the domains, known inhibitors, and K_{D} values of binding partners (i.e., ligands). Examples of detection assays are also provided in Table 2, which may be used in a screening assay to find ligands for the domains.

TABLE 1
Examples of Protein Families and Their Pharmacological Targets

| | | | EXAMPLES OF | EXAMPLES OF | |
|---|---|---|---|---|---|
| | | ENDOGENOUS | CURRENT | CURRENT | EXAMPLES OF |
| TARGET | TARGET | LIGAND | AGONISTS | ANTAGONISTS | DETECTION |
| FAMILY | EXAMPLE | (MODULATORS) | (ACTIVATORS) | (INHIBITORS) | ASSAYS |
| G-PROTEIN | β₂ adrenergic | epinephrine, | albuterol, | propranolol, | HitHunter, PathHunter |
| COUPLED | receptors | norepinephrine | salbutamol, | butoxamine | (DiscoverX), cAMP |
| RECEPTORS | | | terbutaline, | | assay, Intracellular |
| | | | salmeterol | | calcium flux, TANGO, |
| | | | | | GeneBlazer, ELISA, |
| | | | | | binding assays |
| G-PROTEIN | Muscarinic | Acetylcholine | Acetylcholine, | Scopolamine, | HitHunter, PathHunter |
| COUPLED | receptors | | Pilocarpine | atropine, | (DiscoverX), cAMP |
| RECEPTORS | | | | ipratropium, | assay, Intracellular |
| | | | | caproctamine | calcium flux, TANGO, |
| | | | | | GeneBlazer, ELISA, |
| | | | | | binding assays |
| G-PROTEIN | H1 histamine | histamine | Histamine | diphenhydramine, | HitHunter, PathHunter |
| COUPLED | receptor | | | doxylamine, | (DiscoverX), cAMP |
| RECEPTORS | | | | pyrilamine, | assay, Intracellular |
| | | | | brompheniramine, | calcium flux, TANGO, |
| | | | | chlorpheniramine, | GeneBlazer, ELISA, |
| | | | | Loratadine, | binding assays |
| | | | | Fexofenadine, | |
| | | | | Cetrizine, | |
| | | | | Desoratadine | |
| NUCLEAR | Estrogen | Estriol, estrone, | 17-beta-estradiol, | Tamoxifen, ICI | Hit-hunter (Discoverx), |
| RECEPTORS | receptor | estradiol | Chlorotrianisene, | 164,384, | reporter assays, |
| | | | Dienestrol, | Keoxifene, | TANGO, GeneBlazer, |
| | | | Fosfestrol, | Mepitiostane | ELISA, ligand binding |
| | | | Diethylstilbestrol, | | assays, |
| | | | Zeranol | | |
| VOLTAGE | voltage-gated | | veratridine, | tetrodotoxin, | Intracellular ion flux |
| GATED ION | sodium | | aconitine | saxitoxin, | assays |
| CHANNELS | channels | | | | |
| VOLTAGE | voltage-gated | | BAY K 8644, | ω-conotoxin, | Intracellular ion flux |
| GATED ION | calcium | | CGP 28392 | ω-agatoxins, | assays |
| CHANNELS | channels | | | dihydropyridine, | |
| | | | | nifedipine | |
| LIGAND | kainate | glutamate | kainic acid, | CNQX, | HitHunter, PathHunter |
| GATED ION | receptor | | domoic acid, | LY293558, | (DiscoverX), cAMP |
| CHANNELS | | | LY339434, | LY294486 | assay, Intracellular ion |
| | | | ATPA, | | flux, TANGO, |
| | | | iodowillardiine, | | GeneBlazer, ELISA, |
| | | | (2S,4R)-4- | | ligand binding assays, |
| | | | methylglutamic | | |
| | | | acid | | |
| RECEPTOR | epidermal | epidermal growth | EGF, TGFa, | PD153035, anti- | reporter assays, kinase |
| TYROSINE | growth factor | factor | amphiregulin, | EGFR antibody | assays, CO-IP, BRET, |
| KINASES | receptor | | betacellulin, | C225, | FRET, TANGO, |
| | (EGFR) | | epiregulin, | aeroplysinin-1, | GeneBlazer, HitHunter, |
| | | | neuregulins | AG18, AG82, | PathHunter |
| | | | | AG99, AG112, | (DiscoverX), ELISA |
| | | | | AG213, AG490, | |
| | | | | AG494, AG527, | |
| | | | | AG555, AG556 | |
| GROWTH | Vascular | VEGFR | | Ranibizumab, | Hit-hunter (Discoverx), |
| FACTORS | endothelial | | | bevacizumab, | reporter assays, |
| | growth factor | | | sunitinib, | TANGO, GeneBlazer, |
| | | | | sorafenib, | ELISA, ligand binding |
| | | | | axitinib, | assays, |
| | | | | pazopanib, | |
| | | | | Naphthamides | |
| PROTEASES | Caspase | granzyme B; | Granzyme B, | Z-VAD(OMe)- | caspase assays, |
| | | caspase | caspase | FMK, Z-VAD- | apoptosis assays, |
| | | | | CHO | mitochondrial Dy, CO- |
| | | | | | IP, BRET, FRET, |
| | | | | | TANGO, GeneBlazer, |
| | | | | | HitHunter, PathHunter |
| | | | | | (DiscoverX), ELISA |
| PHOSPHATASES | PP1 | phosphoserine/ | | calyculin A, | protein tyrosine |
| | | threonine residues | | nodularin, | phosphatase assay, CO- |
| | | | | tautomycin | IP, BRET, FRET, |
| | | | | | TANGO, GeneBlazer, |
| | | | | | HitHunter, PathHunter |
| | | | | | (DiscoverX), ELISA |
| PROTEIN | ERK | MEK | | AG126, apigenin, | kinase assay, CO-IP, |
| KINASES | | | | Ste- | BRET, FRET, reporter |
| | | | | MPKKKPTPIQLNP-NH2, | assays, TANGO, |
| | | | | H-GYGRKKRRQRRR-G- | GeneBlazer, HitHunter, |
| | | | | MPKKKPTPIQLNP- | PathHunter |
| | | | | NH2, PD98059, | (DiscoverX) |
| | | | | U0126, | |
| MISC | Adenylate | G proteins, calcium | bordetella | NKY80, 2′,3′- | BRET, FRET, calcium |
| ENZYMES | Cyclase | | pertussis, cholera | Dideoxyadenosine, | flux assays, cAMP |
| | | | toxin, forskolin | 2′,5′- | assays, TANGO, |
| | | | | Dideoxyadenosine, | GeneBlazer, HitHunter, |
| | | | | SQ22536, | PathHunter |
| | | | | MDL-12330A | (DiscoverX) |
| MISC | Acetylcholines | | | Caproctamine, | Acetylcholinesterase |
| ENZYMES | terase | | | Metrifonate, | Assay, Amplex Red, |
| | | | | Physostigmine, | Ellman method, HPLC |
| | | | | Galantamine, | |
| | | | | Dyflos, | |
| | | | | Neostigmine | |
| BIOACTIVE | Ceramide | sphingomyelin | TNF Fas | fumonisin B | TLC lipid charring, |
| LIPIDS | | | ligand, 1,25 | | diacylglycerol kinase |
| | | | dihydroxy | | labeling in vitro |
| | | | vitamin D, | | |
| | | | interferon | | |
| CYTOKINES | IL2 | IL2R | BAY 50-4798, | daclizumab, | TANGO, GeneBlazer, |
| | | | P1-30, SP4206 | basiliximab, | HitHunter, PathHunter |
| | | | | SP4206 | (DiscoverX), IL2 |
| | | | | | dependent mouse |
| | | | | | CTLL cell line, ELISA |
| MISC | BCLXL | BAD | | BH3I-1, A- | TANGO, GeneBlazer, |
| PROTEINS | | | | 371191, ABT-737 | HitHunter, PathHunter |
| | | | | | (DiscoverX), CO-IP, |
| | | | | | BRET, FRET, ELISA |
| MISC | p53 | MDM2, JNK1-3, | PRIMA-1, | Pifithrin-α | caspase assays, |
| PROTEINS | | ERK1-2, p38 | MIRA-1, RITA, | | apoptosis assays, |
| | | MAPK, ATR, | | | mitochondrial Dy, CO- |
| | | ATM, Chk1, Chk2, | | | IP, BRET, FRET, |
| | | DNA-PK, CAK | | | TANGO, GeneBlazer, |
| | | | | | HitHunter, PathHunter |
| | | | | | (DiscoverX), ELISA |
| MISC | Tubulin | tubulin | | ALB109564, | kinase assay, CO-IP, |
| PROTEINS | | | | ABT-751, | BRET, FRET, reporter |
| | | | | D24851, D64131, | assays, TANGO, |
| | | | | benomyl, | GeneBlazer, - |
| | | | | estramustine, | arrestin(DiscoverX |
| | | | | LY290181 | |
| MISC | -amyloid | | | L 1,10- | Stagnant Amyloid |
| PROTEINS | | | | phenanthroline | Fibril Formation |
| | | | | derivatives, | Assay, amyloid |
| | | | | KLVFF, LVFFA, | fibrillization assay |
| | | | | Memoquin, SLF- | |
| | | | | CR | |
| MISC | thymidylate | | | raltitrexed, | caspase assays, |
| PROTEINS | synthase | | | pemetrexed, | apoptosis assays, |
| | | | | nolatrexed, | mitochondrial Dy, CO- |
| | | | | ZD9331, | IP, BRET, FRET, |
| | | | | GS7904L, | TANGO, GeneBlazer, |
| | | | | fluorouracil | HitHunter, PathHunter |
| | | | | | (DiscoverX), ELISA |
| UBIQUITIN | MDM2 | p53 | | trans-4-Iodo, 4′- | TANGO, GeneBlazer, |
| LIGASES | | | | boranyl-chalcone, | HitHunter, PathHunter |
| | | | | Nutlins, MI-219, | (DiscoverX), CO-IP, |
| | | | | MI-63, RITA, | BRET, FRET, ELISA, |
| | | | | HLI98 | reporter assay |
| VIRAL | HPV E2 | HPV E1 | | indandiones, | E2 displacement assay, |
| REGULATORS | | | | podophyllotoxin | TANGO, GeneBlazer, |
| | | | | | HitHunter, PathHunter |
| | | | | | (DiscoverX), CO-IP, |
| | | | | | BRET, FRET, ELISA, |
| | | | | | reporter assay |
| BACTERIAL | ZipA | FtsZ | | substituted 3-(2- | TANGO, GeneBlazer, |
| CELL | | | | indolyl)piperidines, | HitHunter, PathHunter |
| DIVISION | | | | 2-phenyl indoles | (DiscoverX), CO-IP, |
| PROTEINS | | | | | BRET, FRET, ELISA, |
| | | | | | reporter assay, |
| | | | | | polarization |
| | | | | | competition assay, |
| CYTOKINES | TNF | TNFR | | infliximab, | TANGO, GeneBlazer, |
| | | | | adalimumab, | HitHunter, PathHunter |
| | | | | etanercept | (DiscoverX), CO-IP, |
| | | | | | BRET, FRET, ELISA, |
| SCAFFOLD | JIP1 | JNK | | BI-78D3, TIJIP | TANGO, GeneBlazer, |
| PROTEINS | | | | | HitHunter, PathHunter |
| | | | | | (DiscoverX), CO-IP, |
| | | | | | BRET, FRET, ELISA, |
| | | | | | kinase assay |
| DNA REPAIR | PARP | | | INO-1001, | TANGO, GeneBlazer, |
| | | | | AG014699, BS- | HitHunter, PathHunter |
| | | | | 201, AZD2281, | (DiscoverX), CO-IP, |
| | | | | BS-401 | BRET, FRET, ELISA, |
| RIBOSOMES | Antibiotics | ribosomes | | tetracyclins, | cell death assay, |
| | | | | macrolides, | |
| | | | | lincosamides, | |
| | | | | streptogramins | |
| HISTONE | HDAC1 | | | suberoylanilide | TANGO, GeneBlazer, |
| DEACETYLASES | | | | hydroxamic acid, | HitHunter, PathHunter |
| | | | | trichostatin A, | (DiscoverX), CO-IP, |
| | | | | LBH589 | BRET, FRET, ELISA, |
| APOPTOSIS | XIAP | SMAC/DIABLO, | | SM102-SM130 | CO-IP, BRET, FRET, |
| REGULATORS | | caspase 3, caspase | | | reporter assays, |
| | | 7, caspase 9 | | | TANGO, GeneBlazer, |
| | | | | | HitHunter, PathHunter |
| | | | | | (DiscoverX), cell death |
| | | | | | assays |
| CHAPERONE | Hsp90 | Cdc37, survivin | | Celastrol, | CO-IP, BRET, FRET, |
| PROTEINS | | | | shepherdin | reporter assays, |
| | | | | | TANGO, GeneBlazer, |
| | | | | | HitHunter, PathHunter |
| | | | | | (DiscoverX), |
| SERINE/ | mTOR | Raptor, | | Rapamycin, | kinase assay, CO-IP, |
| THREONINE | | mLST8/GβL | | caffeine, | BRET, FRET, reporter |
| PROTEIN | | | | farnesylthiosalicylic | assays, TANGO, |
| KINASES | | | | acid, curcumin, | GeneBlazer, HitHunter, |
| | | | | temsirolimus, | PathHunter |
| | | | | everolimus | (DiscoverX) |
| SERINE/ | B-raf & B-raf | K-ras | | PLX4720 | kinase assay, CO-IP, |
| THREONINE- | V600E | | | | BRET, FRET, reporter |
| PROTEIN | | | | | assays, TANGO, |
| KINASES | | | | | GeneBlazer, HitHunter, |
| | | | | | PathHunter |
| | | | | | (DiscoverX), |
| CYCLIN | CDK2 | Cyclin A, cyclin E | | Variolin, | kinase assay, CO-IP, |
| DEPENDENT | | | | Meriolin | BRET, FRET, reporter |
| KINASES | | | | | assays, TANGO, |
| | | | | | GeneBlazer, HitHunter, |
| | | | | | PathHunter |
| | | | | | (DiscoverX), |
| GROWTH | IGF-1R | IGFII | | PQIP | CO-IP, BRET, FRET, |
| FACTOR | | | | | reporter assays, |
| RECEPTORS | | | | | TANGO, GeneBlazer, |
| | | | | | HitHunter, PathHunter |
| | | | | | (DiscoverX), |
| PROTEASOME | 20S | 19S | | Bortezomib, | CO-IP, BRET, FRET, |
| | | | | salinosporamide A, | cell viability |

TABLE 2
Examples of Protein Domains

| | | EXAMPLE OF | | | APPROXIMATE | |
|---|---|---|---|---|---|---|
| | | PROTEIN | EXAMPLES OF | EXAMPLES OF | K_{D} OF | |
| | | CONTAINING | KNOWN | DETECTION | BINDING | |
| DOMAIN | PARTNER | DOMAIN | INHIBITORS | ASSAYS | PARTNERS | |
| SH2 | Phospho-tyrosine | Grb2 | Fmoc-Glu-Tyr-Aib - | Surface | 0.2-11 | μm |
| | residues | | Asn-NH2; Ac- | plasmon | | |
| | | | SpYVNVQ-NH2, | resonance | | |
| | | | macrocycles, | (SPR) | | |
| | | | STATTIC | technology, | | |
| FHA | Phospho-threonine | KIF13B | | | 1-100 | μm |
| | and phospho- | | | | | |
| | tyrosine residues | | | | | |
| 14-3-3 | Phospho-serine | 14-3-3 | R18 | | 7 nM-20 μm | |
| | residues | | | | | |
| WW | ligands containing | Pin1 | Zn(II) | | 6 μm-190 μm | |
| | PpxY, Proline-rich | | Dipicolylamine- | | | |
| | sequences | | based artificial | | | |
| | | | receptors | | | |
| WD40 | | Apaf-1 | | | 1 | μm |
| MH2 | phospho-serine | SMAD2 | | | 240 | nM |
| | residues | | | | | |
| BROMO | acetylated lysine | CBP | | | 1 μm-4 mM | |
| | residues | | | | | |
| UBA | mono-, di-, tri-, and | HHR23A | | | 6 μm-2.35 mM | |
| | tetra-ubiquitin | | | | | |
| PTB | Phospho-tyrosine | IRS-1 | LSNPTX-NH2, | PTB domain | 160 nM-10 μm | |
| | residues, Asn-Pro-X- | | LYASSNOAX- | binding | | |
| | Tyr motifs | | NH2, | assays | | |
| | | | LYASSNPAX-NH2 | | | |
| SH3 | Proline-rich peptides | Grb2 | Peptidimer-c, | | 1-500 | μm |
| | with consensus Pro- | | VPPPVPPRRR, | | | |
| | X-X-Pro, | | (VPPPVPPRRR)2K) | | | |
| EVH1 | FPxΦP motifs, | ActA | | | 10-50 | μm |
| | PPxxF motifs | | | | | |
| GYF | proline-rich | CDBP2 | | | 10-160 | μm |
| | sequences, | | | | | |
| VHS | | TOM1 | | | 11-50 | μm |
| PDZ | PDZ, Val-COOH | MNT1 | NSC668036, FJ9 | | 1-500 | μm |
| PUF | RNA | PUM1 | | | 10-100 | nM |
| TUBBY | DNA, | TULP1 | | | | |
| | phosphotidylinositol | | | | | |
| SAM | | CNK | | | 71 nM-1 μm | |
| DD | DD | FADD | | | | |
| CARD | CARD | Apaf-1 | | | 1.4 | μm |
| PyD | PyD | Pyrin | | | 4 | μm |
| PB1 | PB1 | Bem1 | | | 4-500 | nM |
| BRCT | BRCT | BRCA1 | | | 113 nM-6 μm | |
| PH | phosphatidylinositol- | AKT1 | NSC 348900, | | 1.76 nM-350 μm | |
| | 4,5-bisphosphate, | | perifosine, SH5, | | | |
| | PI-3, 4-P2 or PI- | | SH23, SH24, SH25, | | | |
| | 3,4,5-P3 | | ml14, ml15, ml16 | | | |
| FYVE | Phosphatidylinositol | SARA | | | 50 nM-140 μm | |
| | 3-phosphate, zinc | | | | | |
| C1 | phorbol esters, | PKC isoforms | | | 0.58-800 | nM |
| | diacylglycerol | | | | | |

| | | | |
|---|---|---|---|
| FERM | PI(3)P, PI(4)P, | PTLP1 | 200 nM-30 μm |
| | PI(5)P, IP3, | | |
| C2 | Calcium, acidic | Nedd4 | 250 nM-94 μm |
| | phospholipids | | |
| PX | PI(3,4)P2, PI(3)P, | CISK | 1.8 nM-50 μm |
| | PI(3,5)P2, PI(4)P, | | |
| | PI(5)P, PI(3,4,5)P3, | | |
| | PI(4,5)P2 | | |
| ENTH | PtdIns(4,5)P2, | Epsin1 | 98 nM-1 μm |
| | PtdIns(1,4,5)P3, | | |
| | PI(3,4)P2; PI(3,5)P2 | | |

A pharmacophore is typically an arrangement of the substituents of a moiety that confers biochemical or pharmacological effects. In some embodiments, identification of a pharmacophore may be facilitated by knowing the structure of the ligand in association with a target biomolecule. In some cases, pharmacophores may be moieties derived from molecules previously known to bind to target biomolecules (e.g., proteins), fragments identified, for example, through NMR or crystallographic screening efforts, molecules that have been discovered to bind to target proteins after performing high-throughput screening of natural products libraries, previously synthesized commercial or non-commercial combinatorial compound libraries, or molecules that are discovered to bind to target proteins by screening of newly synthesized combinatorial libraries. Since most pre-existing combinatorial libraries are limited in the structural space and diversity that they encompass, newly synthesized combinatorial libraries may include molecules that are based on a variety of scaffolds.

Additionally pharmacophores may be derived from traditional approaches such as fragment based drug design and structure based drug design. Those skilled in the art will recognize that any pharmacophore including pre-existing pharmacophores such as approved drugs are amenable to be designed as monomers through the incorporation of the appropriate linker elements and connector elements. For example, previously approved drugs that have poor efficacy due to a low affinity for a first macromolecular target may be utilized as a pharmacophore component of a first monomer which when combined with a pharmacophore of a second monomer that also binds the first macromolecular target or a second macromolecular target that interacts with the first macromolecular target results in enhanced binding and, in some cases, higher efficacy. Likewise, previously approved drugs that have low efficacy as a result of size, molecular weight or other physicochemical attributes that reduce the cellular uptake of the drug may be amenable to being converted into one or more monomers that bear the appropriate pharmacophoric elements, such that each monomer has physicochemical attributes that allow for increased cellular uptake.

In some embodiments, a ligand moiety (e.g., a pharmacophore) may have a molecular weight between 50 Da and 2000 Da, in some embodiments between 50 Da and 1500 Da, in some embodiments, between 50 Da and 1000 Da, and in some embodiments, between 50 Da and 500 Da. In certain embodiments, a ligand moiety may have a molecular weight of less than 2000 Da, in some embodiments, less than 1000 Da, and in some embodiments less than 500 Da.

In certain embodiments, the compound utilized by one or more of the foregoing methods is one of the generic, subgeneric, or specific compounds described herein.

Disclosed compositions may be administered to patients (animals and humans) in need of such treatment in dosages that will provide optimal pharmaceutical efficacy. It will be appreciated that the dose required for use in any particular application will vary from patient to patient, not only with the particular compound or composition selected, but also with the route of administration, the nature of the condition being treated, the age and condition of the patient, concurrent medication or special diets then being followed by the patient, and other factors which those skilled in the art will recognize, with the appropriate dosage ultimately being at the discretion of the attendant physician. For treating clinical conditions and diseases noted above, a compound may be administered orally, subcutaneously, topically, parenterally, by inhalation spray or rectally in dosage unit formulations containing conventional non-toxic pharmaceutically acceptable carriers, adjuvants, and vehicles. Parenteral administration may include subcutaneous injections, intravenous or intramuscular injections, or infusion techniques.

Treatment can be continued for as long or as short a period as desired. The compositions may be administered on a regimen of, for example, one to four or more times per day. A suitable treatment period can be, for example, at least about one week, at least about two weeks, at least about one month, at least about six months, at least about 1 year, or indefinitely. A treatment period can terminate when a desired result, for example a partial or total alleviation of symptoms, is achieved.

In another aspect, pharmaceutical compositions comprising monomers, dimers, and/or multimers as disclosed herein formulated together with a pharmaceutically acceptable carrier provided. In particular, the present disclosure provides pharmaceutical compositions comprising monomers, dimers, and/or multimers as disclosed herein formulated together with one or more pharmaceutically acceptable carriers. These formulations include those suitable for oral, rectal, topical, buccal, parenteral (e.g., subcutaneous, intramuscular, intradermal, or intravenous) rectal, vaginal, or aerosol administration, although the most suitable form of administration in any given case will depend on the degree and severity of the condition being treated and on the nature of the particular compound being used. For example, disclosed compositions may be formulated as a unit dose, and/or may be formulated for oral or subcutaneous administration.

Exemplary pharmaceutical compositions may be used in the form of a pharmaceutical preparation, for example, in solid, semisolid, or liquid form, which contains one or more of the compounds, as an active ingredient, in admixture with an organic or inorganic carrier or excipient suitable for external, enteral, or parenteral applications. The active ingredient may be compounded, for example, with the usual non-toxic, pharmaceutically acceptable carriers for tablets, pellets, capsules, suppositories, solutions, emulsions, suspensions, and any other form suitable for use. The active object compound is included in the pharmaceutical composition in an amount sufficient to produce the desired effect upon the process or condition of the disease.

For preparing solid compositions such as tablets, the principal active ingredient may be mixed with a pharmaceutical carrier, e.g., conventional tableting ingredients such as corn starch, lactose, sucrose, sorbitol, talc, stearic acid, magnesium stearate, dicalcium phosphate or gums, and other pharmaceutical diluents, e.g., water, to form a solid preformulation composition containing a homogeneous mixture of a compound, or a non-toxic pharmaceutically acceptable salt thereof. When referring to these preformulation compositions as homogeneous, it is meant that the active ingredient is dispersed evenly throughout the composition so that the composition may be readily subdivided into equally effective unit dosage forms such as tablets, pills and capsules.

In solid dosage forms for oral administration (capsules, tablets, pills, dragees, powders, granules and the like), the subject composition is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, acetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the subject composition moistened with an inert liquid diluent. Tablets, and other solid dosage forms, such as dragees, capsules, pills and granules, may optionally be scored or prepared with coatings and shells, such as enteric coatings and other coatings well known in the pharmaceutical-formulating art.

Compositions for inhalation or insufflation include solutions and suspensions in pharmaceutically acceptable, aqueous or organic solvents, or mixtures thereof, and powders. Liquid dosage forms for oral administration include pharmaceutically acceptable emulsions, microemulsions, solutions, suspensions, syrups and elixirs. In addition to the subject composition, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, cyclodextrins and mixtures thereof

Suspensions, in addition to the subject composition, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof

Formulations for rectal or vaginal administration may be presented as a suppository, which may be prepared by mixing a subject composition with one or more suitable non-irritating excipients or carriers comprising, for example, cocoa butter, polyethylene glycol, a suppository wax or a salicylate, and which is solid at room temperature, but liquid at body temperature and, therefore, will melt in the body cavity and release the active agent.

Dosage forms for transdermal administration of a subject composition includes powders, sprays, ointments, pastes, creams, lotions, gels, solutions, patches and inhalants. The active component may be mixed under sterile conditions with a pharmaceutically acceptable carrier, and with any preservatives, buffers, or propellants which may be required.

The ointments, pastes, creams and gels may contain, in addition to a subject composition, excipients, such as animal and vegetable fats, oils, waxes, paraffins, starch, tragacanth, cellulose derivatives, polyethylene glycols, silicones, bentonites, silicic acid, talc and zinc oxide, or mixtures thereof

Powders and sprays may contain, in addition to a subject composition, excipients such as lactose, talc, silicic acid, aluminum hydroxide, calcium silicates and polyamide powder, or mixtures of these substances. Sprays may additionally contain customary propellants, such as chlorofluorohydrocarbons and volatile unsubstituted hydrocarbons, such as butane and propane.

Compositions and compounds may alternatively be administered by aerosol. This is accomplished by preparing an aqueous aerosol, liposomal preparation or solid particles containing the compound. A non-aqueous (e.g., fluorocarbon propellant) suspension could be used. Sonic nebulizers may be used because they minimize exposing the agent to shear, which may result in degradation of the compounds contained in the subject compositions. Ordinarily, an aqueous aerosol is made by formulating an aqueous solution or suspension of a subject composition together with conventional pharmaceutically acceptable carriers and stabilizers. The carriers and stabilizers vary with the requirements of the particular subject composition, but typically include non-ionic surfactants (Tweens, Pluronics, or polyethylene glycol), innocuous proteins like serum albumin, sorbitan esters, oleic acid, lecithin, amino acids such as glycine, buffers, salts, sugars, or sugar alcohols. Aerosols generally are prepared from isotonic solutions.

Pharmaceutical compositions suitable for parenteral administration comprise a subject composition in combination with one or more pharmaceutically-acceptable sterile isotonic aqueous or non-aqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and non-aqueous carriers which may be employed in the pharmaceutical compositions include water, ethanol, polyols (such as glycerol, propylene glycol, polyethylene glycol, and the like), and suitable mixtures thereof, vegetable oils, such as olive oil, and injectable organic esters, such as ethyl oleate and cyclodextrins. Proper fluidity may be maintained, for example, by the use of coating materials, such as lecithin, by the maintenance of the required particle size in the case of dispersions, and by the use of surfactants

In another aspect, enteral pharmaceutical formulations including a disclosed pharmaceutical composition comprising monomers, dimers, and/or multimers, an enteric material; and a pharmaceutically acceptable carrier or excipient thereof are provided. Enteric materials refer to polymers that are substantially insoluble in the acidic environment of the stomach, and that are predominantly soluble in intestinal fluids at specific pHs. The small intestine is the part of the gastrointestinal tract (gut) between the stomach and the large intestine, and includes the duodenum, jejunum, and ileum. The pH of the duodenum is about 5.5, the pH of the jejunum is about 6.5 and the pH of the distal ileum is about 7.5. Accordingly, enteric materials are not soluble, for example, until a pH of about 5.0, of about 5.2, of about 5.4, of about 5.6, of about 5.8, of about 6.0, of about 6.2, of about 6.4, of about 6.6, of about 6.8, of about 7.0, of about 7.2, of about 7.4, of about 7.6, of about 7.8, of about 8.0, of about 8.2, of about 8.4, of about 8.6, of about 8.8, of about 9.0, of about 9.2, of about 9.4, of about 9.6, of about 9.8, or of about 10.0. Exemplary enteric materials include cellulose acetate phthalate (CAP), hydroxypropyl methylcellulose phthalate (HPMCP), polyvinyl acetate phthalate (PVAP), hydroxypropyl methylcellulose acetate succinate (HPMCAS), cellulose acetate trimellitate, hydroxypropyl methylcellulose succinate, cellulose acetate succinate, cellulose acetate hexahydrophthalate, cellulose propionate phthalate, cellulose acetate maleat, cellulose acetate butyrate, cellulose acetate propionate, copolymer of methylmethacrylic acid and methyl methacrylate, copolymer of methyl acrylate, methylmethacrylate and methacrylic acid, copolymer of methylvinyl ether and maleic anhydride (Gantrez ES series), ethyl methyacrylate-methylmethacrylate-chlorotrimethylammonium ethyl acrylate copolymer, natural resins such as zein, shellac and copal collophorium, and several commercially available enteric dispersion systems (e. g., Eudragit L30D55, Eudragit FS30D, Eudragit L100, Eudragit 5100, Kollicoat EMM30D, Estacryl 30D, Coateric, and Aquateric). The solubility of each of the above materials is either known or is readily determinable in vitro. The foregoing is a list of possible materials, but one of skill in the art with the benefit of the disclosure would recognize that it is not comprehensive and that there are other enteric materials that may be used.

Advantageously, kits are provided containing one or more compositions each including the same or different monomers. Such kits include a suitable dosage form such as those described above and instructions describing the method of using such dosage form to treat a disease or condition. The instructions would direct the consumer or medical personnel to administer the dosage form according to administration modes known to those skilled in the art. Such kits could advantageously be packaged and sold in single or multiple kit units. An example of such a kit is a so-called blister pack. Blister packs are well known in the packaging industry and are being widely used for the packaging of pharmaceutical unit dosage forms (tablets, capsules, and the like). Blister packs generally consist of a sheet of relatively stiff material covered with a foil of a preferably transparent plastic material. During the packaging process recesses are formed in the plastic foil. The recesses have the size and shape of the tablets or capsules to be packed. Next, the tablets or capsules are placed in the recesses and the sheet of relatively stiff material is sealed against the plastic foil at the face of the foil which is opposite from the direction in which the recesses were formed. As a result, the tablets or capsules are sealed in the recesses between the plastic foil and the sheet. Preferably the strength of the sheet is such that the tablets or capsules can be removed from the blister pack by manually applying pressure on the recesses whereby an opening is formed in the sheet at the place of the recess. The tablet or capsule can then be removed via said opening.

It may be desirable to provide a memory aid on the kit, e.g., in the form of numbers next to the tablets or capsules whereby the numbers correspond with the days of the regimen which the tablets or capsules so specified should be ingested. Another example of such a memory aid is a calendar printed on the card, e.g., as follows “First Week, Monday, Tuesday, . . . etc. . . . Second Week, Monday, Tuesday, . . . ” etc. Other variations of memory aids will be readily apparent. A “daily dose” can be a single tablet or capsule or several pills or capsules to be taken on a given day. Also, a daily dose of a first compound can consist of one tablet or capsule while a daily dose of the second compound can consist of several tablets or capsules and vice versa. The memory aid should reflect this.

Also contemplated herein are methods and compositions that include a second active agent, or administering a second active agent.

Also contemplated herein are methods and compositions that include a second active agent, or administering a second active agent.

Certain terms employed in the specification, examples, and appended claims are collected here. These definitions should be read in light of the entirety of the disclosure and understood as by a person of skill in the art. Unless defined otherwise, all technical and scientific terms used herein have the same meaning as commonly understood by a person of ordinary skill in the art.

### DEFINITIONS

In some embodiments, the compounds, as described herein, may be substituted with any number of substituents or functional moieties. In general, the term “substituted” whether preceded by the term “optionally” or not, and substituents contained in formulas, refer to the replacement of hydrogen radicals in a given structure with the radical of a specified substituent.

In some instances, when more than one position in any given structure may be substituted with more than one substituent selected from a specified group, the substituent may be either the same or different at every position.

As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. In some embodiments, heteroatoms such as nitrogen may have hydrogen substituents and/or any permissible substituents of organic compounds described herein which satisfy the valencies of the heteroatoms. Non-limiting examples of substituents include acyl; aliphatic; heteroaliphatic; aryl; heteroaryl; arylalkyl; heteroarylalkyl; alkoxy; cycloalkoxy; heterocyclylalkoxy; heterocyclyloxy; heterocyclyloxyalkyl; alkenyloxy; alkynyloxy; aryloxy; heteroalkoxy; heteroaryloxy; alkylthio; arylthio; heteroalkylthio; heteroarylthio; oxo; —F; —Cl; —Br; —I; —OH; —NO₂; —CN; —SCN; —SRₓ; —CF₃; —CH₂CF₃; —CHCl₂; —CH₂OH; —CH₂CH₂OH; —CH₂NH₂; —CH₂SO₂CH₃; —ORₓ, —C(O)Rₓ; —CO₂(Rₓ); —C(O)N(Rₓ)₂; —OC(O)Rₓ; —OCO₂Rₓ; —OC(O)N(Rₓ)₂; —N(Rₓ)₂; —SORₓ; —S(O)₂Rₓ; —NRₓC(O)Rₓ; or —C(Rₓ)₃; wherein each occurrence of Rₓ independently includes, but is not limited to, hydrogen, aliphatic, heteroaliphatic, aryl, heteroaryl, arylalkyl, or heteroarylalkyl, wherein any of the aliphatic, heteroaliphatic, arylalkyl, or heteroarylalkyl substituents described above and herein may be substituted or unsubstituted, branched or unbranched, cyclic or acyclic, and wherein any of the aryl or heteroaryl substituents described above and herein may be substituted or unsubstituted. Furthermore, the compounds described herein are not intended to be limited in any manner by the permissible substituents of organic compounds. In some embodiments, combinations of substituents and variables described herein may be preferably those that result in the formation of stable compounds. The term “stable,” as used herein, refers to compounds which possess stability sufficient to allow manufacture and which maintain the integrity of the compound for a sufficient period of time to be detected and preferably for a sufficient period of time to be useful for the purposes detailed herein.

The term “acyl,” as used herein, refers to a moiety that includes a carbonyl group. In some embodiments, an acyl group may have a general formula selected from —C(O)Rₓ; —CO₂(Rₓ); —C(O)N(Rₓ)₂; —OC(O)Rₓ; —OCO₂Rₓ; and —OC(O)N(Rₓ)₂; wherein each occurrence of Rₓ independently includes, but is not limited to, hydrogen, aliphatic, heteroaliphatic, aryl, heteroaryl, arylalkyl, or heteroarylalkyl, wherein any of the aliphatic, heteroaliphatic, arylalkyl, or heteroarylalkyl substituents described above and herein may be substituted or unsubstituted, branched or unbranched, cyclic or acyclic, and wherein any of the aryl or heteroaryl substituents described above and herein may be substituted or unsubstituted.

The term “aliphatic,” as used herein, includes both saturated and unsaturated, straight chain (i.e., unbranched), branched, acyclic, cyclic, or polycyclic aliphatic hydrocarbons, which are optionally substituted with one or more functional groups. As will be appreciated by one of ordinary skill in the art, “aliphatic” is intended herein to include, but is not limited to, alkyl, alkenyl, alkynyl, cycloalkyl, cycloalkenyl, and cycloalkynyl moieties.

The term “heteroaliphatic,” as used herein, refers to aliphatic moieties that contain one or more oxygen, sulfur, nitrogen, phosphorus, or silicon atoms, e.g., in place of carbon atoms. Heteroaliphatic moieties may be branched, unbranched, cyclic or acyclic and include saturated and unsaturated heterocycles such as morpholino, pyrrolidinyl, etc. In certain embodiments, heteroaliphatic moieties are substituted by independent replacement of one or more of the hydrogen atoms thereon with one or more moieties including, but not limited to acyl; aliphatic; heteroaliphatic; aryl; heteroaryl; arylalkyl; heteroarylalkyl; alkoxy; cycloalkoxy; heterocyclylalkoxy; heterocyclyloxy; heterocyclyloxyalkyl; alkenyloxy; alkynyloxy; aryloxy; heteroalkoxy; heteroaryloxy; alkylthio; arylthio; heteroalkylthio; heteroarylthio; oxo; —F; —Cl; —Br; —I; —OH; —NO₂; —CN; —SCN; —SRₓ; —CF₃; —CH₂CF₃; —CHCl₂; —CH₂OH; —CH₂CH₂OH; —CH₂NH₂; —CH₂SO₂CH₃; —ORₓ, —C(O)Rₓ; —OC₂(Rₓ); —C(O)N(Rₓ)₂; —OC(O)Rₓ; —OCO₂Rₓ; —OC(O)N(Rₓ)₂; —N(Rₓ)₂; —SORₓ; —S(O)₂Rₓ; —NRₓC(O)Rₓ; or —C(Rₓ)₃; wherein each occurrence of Rₓ independently includes, but is not limited to, hydrogen, aliphatic, heteroaliphatic, aryl, heteroaryl, arylalkyl, or heteroarylalkyl, wherein any of the aliphatic, heteroaliphatic, arylalkyl, or heteroarylalkyl substituents described above and herein may be substituted or unsubstituted, branched or unbranched, cyclic or acyclic, and wherein any of the aryl or heteroaryl substituents described above and herein may be substituted or unsubstituted.

In general, the terms “aryl” and “heteroaryl,” as used herein, refer to stable mono- or polycyclic, heterocyclic, polycyclic, and polyheterocyclic unsaturated moieties having preferably 3-14 carbon atoms, each of which may be substituted or unsubstituted. Substituents include, but are not limited to, any of the previously mentioned substituents, i.e., the substituents recited for aliphatic moieties, or for other moieties as disclosed herein, resulting in the formation of a stable compound. In certain embodiments, aryl refers to a mono- or bicyclic carbocyclic ring system having one or two aromatic rings including, but not limited to, phenyl, naphthyl, tetrahydronaphthyl, indanyl, indenyl, and the like. In certain embodiments, the term heteroaryl, as used herein, refers to a cyclic aromatic radical having from five to ten ring atoms of which one ring atom is selected from the group consisting of S, O, and N; zero, one, or two ring atoms are additional heteroatoms independently selected from the group consisting of S, O, and N; and the remaining ring atoms are carbon, the radical being joined to the rest of the molecule via any of the ring atoms, such as, for example, pyridyl, pyrazinyl, pyrimidinyl, pyrrolyl, pyrazolyl, imidazolyl, thiazolyl, oxazolyl, isooxazolyl, thiadiazolyl, oxadiazolyl, thiophenyl, furanyl, quinolinyl, isoquinolinyl, and the like.

It will be appreciated that aryl and heteroaryl groups can be unsubstituted or substituted, wherein substitution includes replacement of one, two, three, or more of the hydrogen atoms thereon independently with any one or more of the following moieties including, but not limited to: aliphatic; heteroaliphatic; aryl; heteroaryl; arylalkyl; heteroarylalkyl; alkoxy; cycloalkoxy; heterocyclylalkoxy; heterocyclyloxy; heterocyclyloxyalkyl; alkenyloxy; alkynyloxy; aryloxy; heteroalkoxy; heteroaryloxy; alkylthio; arylthio; heteroalkylthio; heteroarylthio; oxo; —F; —Cl; —Br; —I; —OH; —NO₂; —CN; —CF₃; —CH₂CF₃; —CHCl₂; —CH₂OH; —CH₂CH₂OH; —CH₂NH₂; —CH₂SO₂CH₃; —C(O)Rₓ; —OC₂(Rₓ); —CON(Rₓ)₂; —OC(O)Rₓ; —OCO₂Rₓ; —OCON(Rₓ)₂; —N(Rₓ)₂; —S(O)₂Rₓ; —NRₓ(CO)Rₓ, wherein each occurrence of Rₓ independently includes, but is not limited to, hydrogen, aliphatic, heteroaliphatic, aryl, heteroaryl, arylalkyl, or heteroarylalkyl, wherein any of the aliphatic, heteroaliphatic, arylalkyl, or heteroarylalkyl substituents described above and herein may be substituted or unsubstituted, branched or unbranched, cyclic or acyclic, and wherein any of the aryl or heteroaryl substituents described above and herein may be substituted or unsubstituted. Additional examples of generally applicable substituents are illustrated by the specific embodiments shown in the Examples that are described herein.

The term “heterocyclic,” as used herein, refers to an aromatic or non-aromatic, partially unsaturated or fully saturated, 3- to 10-membered ring system, which includes single rings of 3 to 8 atoms in size and bi- and tri-cyclic ring systems which may include aromatic five- or six-membered aryl or aromatic heterocyclic groups fused to a non-aromatic ring. These heterocyclic rings include those having from one to three heteroatoms independently selected from the group consisting of oxygen, sulfur, and nitrogen, in which the nitrogen and sulfur heteroatoms may optionally be oxidized and the nitrogen heteroatom may optionally be quaternized. In certain embodiments, the term heterocyclic refers to a non-aromatic 5-, 6-, or 7-membered ring or a polycyclic group wherein at least one ring atom is a heteroatom selected from the group consisting of O, S, and N (wherein the nitrogen and sulfur heteroatoms may be optionally oxidized), including, but not limited to, a bi- or tri-cyclic group, comprising fused six-membered rings having between one and three heteroatoms independently selected from the group consisting of the oxygen, sulfur, and nitrogen, wherein (i) each 5-membered ring has 0 to 2 double bonds, each 6-membered ring has 0 to 2 double bonds, and each 7-membered ring has 0 to 3 double bonds, (ii) the nitrogen and sulfur heteroatoms may be optionally oxidized, (iii) the nitrogen heteroatom may optionally be quaternized, and (iv) any of the above heterocyclic rings may be fused to an aryl or heteroaryl ring.

The term “alkenyl” as used herein refers to an unsaturated straight or branched hydrocarbon having at least one carbon-carbon double bond, such as a straight or branched group of 2-6 or 3-4 carbon atoms, referred to herein for example as C₂₋₆alkenyl, and C₃₋₄alkenyl, respectively. Exemplary alkenyl groups include, but are not limited to, vinyl, allyl, butenyl, pentenyl, etc.

The term “alkenyloxy” used herein refers to a straight or branched alkenyl group attached to an oxygen (alkenyl-O). Exemplary alkenoxy groups include, but are not limited to, groups with an alkenyl group of 3-6 carbon atoms referred to herein as C₃₋₆alkenyloxy. Exemplary “alkenyloxy” groups include, but are not limited to allyloxy, butenyloxy, etc.

The term “alkoxy” as used herein refers to a straight or branched alkyl group attached to an oxygen (alkyl-O—). Exemplary alkoxy groups include, but are not limited to, groups with an alkyl group of 1-6 or 2-6 carbon atoms, referred to herein as C₁₋₆alkoxy, and C₂-C₆alkoxy, respectively. Exemplary alkoxy groups include, but are not limited to methoxy, ethoxy, isopropoxy, etc.

The term “alkoxycarbonyl” as used herein refers to a straight or branched alkyl group attached to oxygen, attached to a carbonyl group (alkyl-O—C(O)—). Exemplary alkoxycarbonyl groups include, but are not limited to, alkoxycarbonyl groups of 1-6 carbon atoms, referred to herein as C₁₋₆alkoxycarbonyl. Exemplary alkoxycarbonyl groups include, but are not limited to, methoxycarbonyl, ethoxycarbonyl, t-butoxycarbonyl, etc.

The term “alkynyloxy” used herein refers to a straight or branched alkynyl group attached to an oxygen (alkynyl-O)). Exemplary alkynyloxy groups include, but are not limited to, propynyloxy.

The term “alkyl” as used herein refers to a saturated straight or branched hydrocarbon, for example, such as a straight or branched group of 1-6, 1-4, or 1-3 carbon atoms, referred to herein as C₁₋₆alkyl, C₁₋₄alkyl, and C₁₋₃alkyl, respectively. Exemplary alkyl groups include, but are not limited to, methyl, ethyl, propyl, isopropyl, 2-methyl-1-propyl, 2-methyl-2-propyl, 2-methyl-1-butyl, 3-methyl-1-butyl, 3-methyl-2-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, butyl, isobutyl, t-butyl, pentyl, isopentyl, neopentyl, hexyl, etc.

The term “alkylcarbonyl” as used herein refers to a straight or branched alkyl group attached to a carbonyl group (alkyl-C(O)—). Exemplary alkylcarbonyl groups include, but are not limited to, alkylcarbonyl groups of 1-6 atoms, referred to herein as C₁₋₆alkylcarbonyl groups. Exemplary alkylcarbonyl groups include, but are not limited to, acetyl, propanoyl, isopropanoyl, butanoyl, etc.

The term “alkynyl” as used herein refers to an unsaturated straight or branched hydrocarbon having at least one carbon-carbon triple bond, such as a straight or branched group of 2-6, or 3-6 carbon atoms, referred to herein as C₂₋₆alkynyl, and C₃₋₆alkynyl, respectively. Exemplary alkynyl groups include, but are not limited to, ethynyl, propynyl, butynyl, pentynyl, hexynyl, methylpropynyl, etc.

The term “carbonyl” as used herein refers to the radical —C(O)—.

The term “carboxylic acid” as used herein refers to a group of formula —CO₂H.

The term “cyano” as used herein refers to the radical —CN.

The term “cycloalkoxy” as used herein refers to a cycloalkyl group attached to an oxygen (cycloalkyl-O—).

The term “cycloalkyl” as used herein refers to a monocyclic saturated or partially unsaturated hydrocarbon group of for example 3-6, or 4-6 carbons, referred to herein, e.g., as C₃₋6cycloalkyl or C₄₋₆cycloalkyl and derived from a cycloalkane. Exemplary cycloalkyl groups include, but are not limited to, cyclohexyl, cyclohexenyl, cyclopentyl, cyclobutyl or, cyclopropyl.

The terms “halo” or “halogen” as used herein refer to F, Cl, Br, or I.

The term “heterocyclylalkoxy” as used herein refers to a heterocyclyl-alkyl-O— group.

The term “heterocyclyloxyalkyl” refers to a heterocyclyl-O-alkyl-group.

The term “heterocyclyloxy” refers to a heterocyclyl-O— group.

The term “heteroaryloxy” refers to a heteroaryl-O— group.

The terms “hydroxy” and “hydroxyl” as used herein refers to the radical —OH.

The term “oxo” as used herein refers to the radical ═O.

The term “connector” as used herein to refers to an atom or a collection of atoms optionally used to link interconnecting moieties, such as a disclosed linker and a pharmacophore. Contemplated connectors are generally hydrolytically stable.

“Treating” includes any effect, e.g., lessening, reducing, modulating, or eliminating, that results in the improvement of the condition, disease, disorder and the like.

“Pharmaceutically or pharmacologically acceptable” include molecular entities and compositions that do not produce an adverse, allergic, or other untoward reaction when administered to an animal, or a human, as appropriate. For human administration, preparations should meet sterility, pyrogenicity, general safety and purity standards as required by FDA Office of Biologics standards.

The term “pharmaceutically acceptable carrier” or “pharmaceutically acceptable excipient” as used herein refers to any and all solvents, dispersion media, coatings, isotonic and absorption delaying agents, and the like, that are compatible with pharmaceutical administration. The use of such media and agents for pharmaceutically active substances is well known in the art. The compositions may also contain other active compounds providing supplemental, additional, or enhanced therapeutic functions.

The term “pharmaceutical composition” as used herein refers to a composition comprising at least one compound as disclosed herein formulated together with one or more pharmaceutically acceptable carriers.

“Individual,” “patient,” or “subject” are used interchangeably and include any animal, including mammals, preferably mice, rats, other rodents, rabbits, dogs, cats, swine, cattle, sheep, horses, or primates, and most preferably humans. The compounds can be administered to a mammal, such as a human, but can also be administered to other mammals such as an animal in need of veterinary treatment, e.g., domestic animals (e.g., dogs, cats, and the like), farm animals (e.g., cows, sheep, pigs, horses, and the like) and laboratory animals (e.g., rats, mice, guinea pigs, and the like). The mammal treated is desirably a mammal in which treatment of obesity, or weight loss is desired. “Modulation” includes antagonism (e.g., inhibition), agonism, partial antagonism and/or partial agonism.

In the present specification, the term “therapeutically effective amount” means the amount of the subject compound that will elicit the biological or medical response of a tissue, system, animal, or human that is being sought by the researcher, veterinarian, medical doctor, or other clinician. The compounds are administered in therapeutically effective amounts to treat a disease. Alternatively, a therapeutically effective amount of a compound is the quantity required to achieve a desired therapeutic and/or prophylactic effect, such as an amount which results in weight loss.

The term “pharmaceutically acceptable salt(s)” as used herein refers to salts of acidic or basic groups that may be present in compounds used in the present compositions. Compounds included in the present compositions that are basic in nature are capable of forming a wide variety of salts with various inorganic and organic acids. The acids that may be used to prepare pharmaceutically acceptable acid addition salts of such basic compounds are those that form non-toxic acid addition salts, i.e., salts containing pharmacologically acceptable anions, including but not limited to malate, oxalate, chloride, bromide, iodide, nitrate, sulfate, bisulfate, phosphate, acid phosphate, isonicotinate, acetate, lactate, salicylate, citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate and pamoate (i.e., 1,1′-methylene-bis-(2-hydroxy-3-naphthoate)) salts. Compounds included in the present compositions that are acidic in nature are capable of forming base salts with various pharmacologically acceptable cations. Examples of such salts include alkali metal or alkaline earth metal salts and, particularly, calcium, magnesium, sodium, lithium, zinc, potassium, and iron salts. Compounds included in the present compositions that include a basic or acidic moiety may also form pharmaceutically acceptable salts with various amino acids. The compounds of the disclosure may contain both acidic and basic groups; for example, one amino and one carboxylic acid group. In such a case, the compound can exist as an acid addition salt, a zwitterion, or a base salt.

The compounds of the disclosure may contain one or more chiral centers and/or double bonds and, therefore, exist as stereoisomers, such as geometric isomers, enantiomers or diastereomers. The term “stereoisomers” when used herein consist of all geometric isomers, enantiomers or diastereomers. These compounds may be designated by the symbols “R” or “S,” depending on the configuration of substituents around the stereogenic carbon atom. Various stereoisomers of these compounds and mixtures thereof are encompassed by this disclosure. Stereoisomers include enantiomers and diastereomers. Mixtures of enantiomers or diastereomers may be designated “(±)” in nomenclature, but the skilled artisan will recognize that a structure may denote a chiral center implicitly.

The compounds of the disclosure may contain one or more chiral centers and/or double bonds and, therefore, exist as geometric isomers, enantiomers or diastereomers. The enantiomers and diastereomers may be designated by the symbols “(+),” “(−).” “R” or “S,” depending on the configuration of substituents around the stereogenic carbon atom, but the skilled artisan will recognize that a structure may denote a chiral center implicitly. Geometric isomers, resulting from the arrangement of substituents around a carbon-carbon double bond or arrangement of substituents around a cycloalkyl or heterocyclic ring, can also exist in the compounds. The symbol [symbol] denotes a bond that may be a single, double or triple bond as described herein. Substituents around a carbon-carbon double bond are designated as being in the “Z” or “E” configuration wherein the terms “Z” and “E” are used in accordance with IUPAC standards. Unless otherwise specified, structures depicting double bonds encompass both the “E” and “Z” isomers. Substituents around a carbon-carbon double bond alternatively can be referred to as “cis” or “trans,” where “cis” represents substituents on the same side of the double bond and “trans” represents substituents on opposite sides of the double bond. The arrangement of substituents around a carbocyclic ring can also be designated as “cis” or “trans.” The term “cis” represents substituents on the same side of the plane of the ring and the term “trans” represents substituents on opposite sides of the plane of the ring. Mixtures of compounds wherein the substituents are disposed on both the same and opposite sides of plane of the ring are designated “cis/trans.”

The term “stereoisomers” when used herein consist of all geometric isomers, enantiomers or diastereomers. Various stereoisomers of these compounds and mixtures thereof are encompassed by this disclosure.

Individual enantiomers and diasteriomers of the compounds can be prepared synthetically from commercially available starting materials that contain asymmetric or stereogenic centers, or by preparation of racemic mixtures followed by resolution methods well known to those of ordinary skill in the art. These methods of resolution are exemplified by (1) attachment of a mixture of enantiomers to a chiral auxiliary, separation of the resulting mixture of diastereomers by recrystallization or chromatography and liberation of the optically pure product from the auxiliary, (2) salt formation employing an optically active resolving agent, (3) direct separation of the mixture of optical enantiomers on chiral liquid chromatographic columns or (4) kinetic resolution using stereoselective chemical or enzymatic reagents. Racemic mixtures can also be resolved into their component enantiomers by well known methods, such as chiral-phase gas chromatography or crystallizing the compound in a chiral solvent. Stereoselective syntheses, a chemical or enzymatic reaction in which a single reactant forms an unequal mixture of stereoisomers during the creation of a new stereocenter or during the transformation of a pre-existing one, are well known in the art. Stereoselective syntheses encompass both enantio- and diastereoselective transformations. For examples, see Carreira and Kvaerno, Classics in Stereoselective Synthesis, Wiley-VCH: Weinheim, 2009.

The compounds disclosed herein can exist in solvated as well as unsolvated forms with pharmaceutically acceptable solvents such as water, ethanol, and the like. In one embodiment, the compound is amorphous. In one embodiment, the compound is a polymorph. In another embodiment, the compound is in a crystalline form.

Also embraced are isotopically labeled compounds which are identical to those recited herein, except that one or more atoms are replaced by an atom having an atomic mass or mass number different from the atomic mass or mass number usually found in nature. Examples of isotopes that can be incorporated into the compounds include isotopes of hydrogen, carbon, nitrogen, oxygen, phosphorus, sulfur, fluorine and chlorine, such as ²H, ³H, ¹³C, ¹⁴C, ¹⁵N, ¹⁸O, ¹⁷O, ³¹P, ³²P, ³⁵S, ¹⁸F, ¹⁰B, and ³⁶Cl, respectively. For example, a compound may have one or more H atom replaced with deuterium.

Certain isotopically-labeled disclosed compounds (e.g., those labeled with ³H and ¹⁴C) are useful in compound and/or substrate tissue distribution assays. Tritiated (i.e., ³H) and carbon-14 (i.e., ¹⁴C) isotopes are particularly preferred for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements) and hence may be preferred in some circumstances. Isotopically labeled compounds can generally be prepared by following procedures analogous to those disclosed in the Examples herein by substituting an isotopically labeled reagent for a non-isotopically labeled reagent.

The term “prodrug” refers to compounds that are transformed in vivo to yield a disclosed compound or a pharmaceutically acceptable salt, hydrate or solvate of the compound. The transformation may occur by various mechanisms (such as by esterase, amidase, phosphatase, oxidative and or reductive metabolism) in various locations (such as in the intestinal lumen or upon transit of the intestine, blood, or liver). Prodrugs are well known in the art (for example, see Rautio, Kumpulainen, et al, Nature Reviews Drug Discovery 2008, 7, 255). For example, if a compound or a pharmaceutically acceptable salt, hydrate, or solvate of the compound contains a carboxylic acid functional group, a prodrug can comprise an ester formed by the replacement of the hydrogen atom of the acid group with a group such as (C₁₋₈)alkyl, (C₂₋₁₂)alkanoyloxymethyl, 1-(alkanoyloxy)ethyl having from 4 to 9 carbon atoms, 1-methyl-1-(alkanoyloxy)-ethyl having from 5 to 10 carbon atoms, alkoxycarbonyloxymethyl having from 3 to 6 carbon atoms, 1-(alkoxycarbonyloxy)ethyl having from 4 to 7 carbon atoms, 1-methyl-1-(alkoxycarbonyloxy)ethyl having from 5 to 8 carbon atoms, N-(alkoxycarbonyl)aminomethyl having from 3 to 9 carbon atoms, 1-(N-(alkoxycarbonyl)amino)ethyl having from 4 to 10 carbon atoms, 3-phthalidyl, 4-crotonolactonyl, gamma-butyrolacton-4-yl, di-N,N—(C₁-C₂)alkylamino(C₂-C₃)alkyl (such as β-dimethylaminoethyl), carbamoyl-(C₁-C₂)alkyl, N,N-di(C₁-C₂)alkylcarbamoyl-(C₁-C₂)alkyl and piperidino-, pyrrolidino- or morpholino(C₂-C₃)alkyl.

Similarly, if a compound contains an alcohol functional group, a prodrug can be formed by the replacement of the hydrogen atom of the alcohol group with a group such as (C₁₋₆)alkanoyloxymethyl, 1-((C₁₋₆)alkanoyloxy)ethyl, 1-methyl-1-((C₁₋₆)alkanoyloxy)ethyl (C₁₋₆)alkoxycarbonyloxymethyl, N—(C₁₋₆)alkoxycarbonylaminomethyl, succinoyl, (C₁₋₆)alkanoyl, α-amino(C₁₋₄ alkanoyl, arylacyl and α-aminoacyl, or α-aminoacyl-α-aminoacyl, where each α-aminoacyl group is independently selected from the naturally occurring L-amino acids, P(O)(OH)₂, —P(O)(O(C₁-C₆)alkyl)₂ or glycosyl (the radical resulting from the removal of a hydroxyl group of the hemiacetal form of a carbohydrate).

If a compound incorporates an amine functional group, a prodrug can be formed, for example, by creation of an amide or carbamate, an N-acyloxyakyl derivative, an (oxodioxolenyl)methyl derivative, an N-Mannich base, imine, or enamine. In addition, a secondary amine can be metabolically cleaved to generate a bioactive primary amine, or a tertiary amine can be metabolically cleaved to generate a bioactive primary or secondary amine. For examples, see Simplicio, et al., Molecules 2008, 13, 519 and references therein.

### INCORPORATION BY REFERENCE

All publications and patents mentioned herein, including those items listed below, are hereby incorporated by reference in their entirety for all purposes as if each individual publication or patent was specifically and individually incorporated by reference. In case of conflict, the present application, including any definitions herein, will control.

### EXAMPLES

The compounds described herein can be prepared in a number of ways based on the teachings contained herein and synthetic procedures known in the art. In the description of the synthetic methods described below, it is to be understood that all proposed reaction conditions, including choice of solvent, reaction atmosphere, reaction temperature, duration of the experiment and workup procedures, can be chosen to be the conditions standard for that reaction, unless otherwise indicated. It is understood by one skilled in the art of organic synthesis that the functionality present on various portions of the molecule should be compatible with the reagents and reactions proposed. Substituents not compatible with the reaction conditions will be apparent to one skilled in the art, and alternate methods are therefore indicated. The starting materials for the examples are either commercially available or are readily prepared by standard methods from known materials.

At least some of the compounds identified as “Intermediates” herein are contemplated as active ingredients.

For ease of reading intermediates are provided in Table 3. At least some of the compounds identified as “Intermediates” herein are contemplated as compounds of the invention. Example compounds are provided in Table 4.

TABLE 3
INTERMEDIATES

| | | |
|---|---|---|
| Sr. | | |
| No. | Structure | Compound Name |

Sparsomycin analogues

| | | |
|---|---|---|
| 1. | 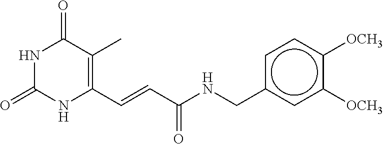 | (E)-N-(3,4-dimethoxybenzyl)-3-(5-methyl-2,6- dioxo-1,2,3,6-tetrahydropyrimidin-4- yl)acrylamide |
| 2. | 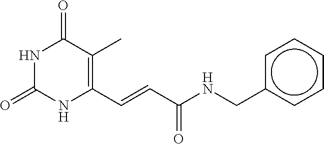 | (E)-N-benzyl-3-(5-methyl-2,6-dioxo-1,2,3,6- tetrahydropyrimidin-4-yl)acrylamide |
| 3. | 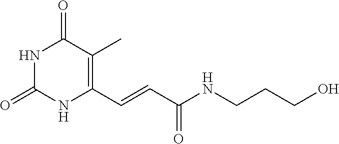 [SMILES: Cc1c(/C=C/C(=O)NCCCO)[nH]c(=O)[nH]c1=O] | (E)-N-(3-hydroxypropyl)-3-(5-methyl-2,6- dioxo-1,2,3,6-tetrahydropyrimidin-4- yl)acrylamide |
| 4. | 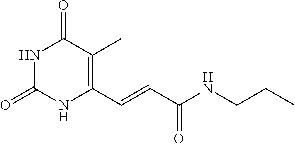 [SMILES: CCCNC(=O)/C=C/c1[nH]c(=O)[nH]c(=O)c1C] | (E)-3-(5-methyl-2,6-dioxo-1,2,3,6- tetrahydropyrimidin-4-yl)-N-propylacrylamide |

Tryptase targets
Method-D

| | | |
|---|---|---|
| 5. | 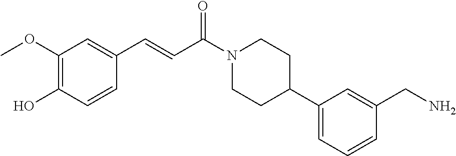 [SMILES: COc1cc(/C=C/C(=O)N2CCC(c3cccc(CN)c3)CC2)ccc1O] | (E)-1-(4-(3-(aminomethyl)phenyl)piperidin-1- yl)-3-(4-hydroxy-3-methoxyphenyl)prop-2-en- 1-one |
| 6. | 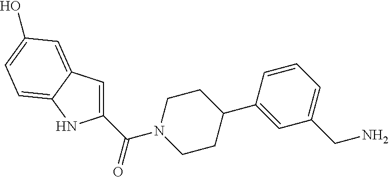 [SMILES: NCc1cccc(C2CCN(C(=O)c3cc4cc(O)ccc4[nH]3)CC2)c1] | (4-(3-(aminomethyl)phenyl)piperidin-1-yl)(5- hydroxy-1H-indol-2-yl)methanone |
| 7. | 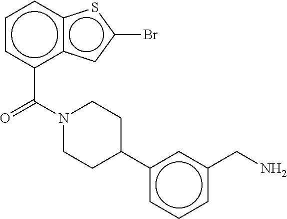 | (4-(3-(aminomethyl)phenyl)piperidin-1-yl)(2- bromobenzo[b]thiophen-4-yl)methanone |
| 8. | 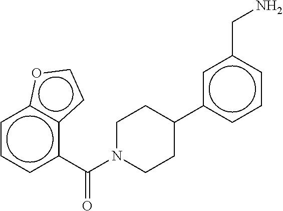 | (4-(3-(aminomethyl)phenyl)piperidin-1- yl)(benzofuran-4-yl)methanone |
| 9. | 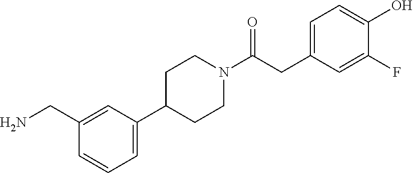 [SMILES: NCc1cccc(C2CCN(C(=O)Cc3ccc(O)c(F)c3)CC2)c1] | 1-(4-(3-(aminomethyl)phenyl)piperidin-1-yl)-2- (3-fluoro-4-hydroxyphenyl)ethanone |
| 10. | 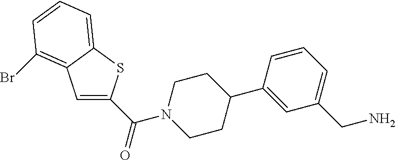 [SMILES: NCc1cccc(C2CCN(C(=O)c3cc4c(Br)cccc4s3)CC2)c1] | (4-(3-(aminomethyl)phenyl)piperidin-1-yl)(4- bromobenzo[b]thiophen-2-yl)methanone |
| 11. | 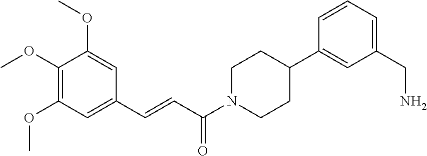 [SMILES: COc1cc(/C=C/C(=O)N2CCC(c3cccc(CN)c3)CC2)cc(OC)c1OC] | (E)-1-(4-(3-(aminomethyl)phenyl)piperidin-1- yl)-3-(3,4,5-trimethoxyphenyl)prop-2-en-1-one |
| 12. | 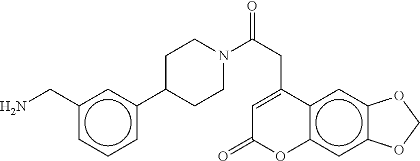 | 8-(2-(4-(3-(aminomethyl)phenyl)piperidin-1- yl)-2-oxoethyl)-6H-[1,3]dioxolo[4,5- g]chromen-6-one |

Tryptase targets
Method-I

| | | |
|---|---|---|
| 13. | 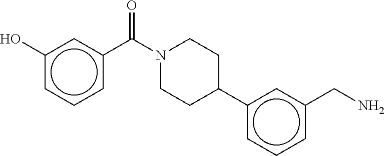 | (4-(3-(aminomethyl)phenyl)piperidin-1-yl)(3- hydroxyphenyl)methanone |

Uncategorized targets

| | | |
|---|---|---|
| 14. | 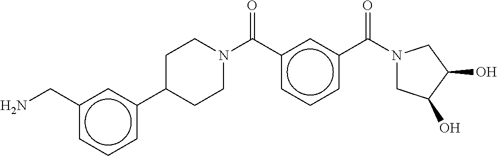 | (4-(3-(aminomethyl)phenyl)piperidin-1-yl)(3- ((3S,4R)-3,4-dihydroxypyrrolidine-1- carbonyl)phenyl)methanone |
| 15. | 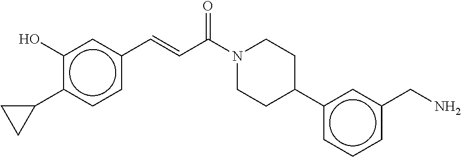 | (E)-1-(4-(3-(aminomethyl)phenyl)piperidin-1- yl)-3-(4-cyclopropyl-3-hydroxyphenyl)prop-2- en-1-one |

TABLE 4
EXAMPLES INDEX

| | | | |
|---|---|---|---|
| Sr. | Compound | | |
| No. | code | Structure | Shipment date |

Linezolid analogues

| | | | |
|---|---|---|---|
| 16. | | | |
| 17. | Lz-NA-1 | 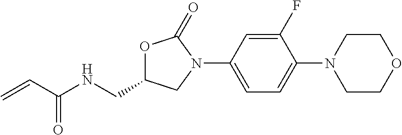 [SMILES: C=CC(=O)NC[C@H]1CN(c2ccc(N3CCOCC3)c(F)c2)C(=O)O1] | 8-Oct-2010 |
| 18. | Lz-NA-2 | 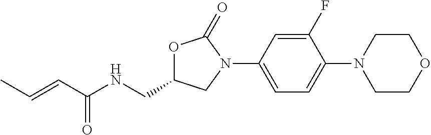 [SMILES: C/C=C/C(=O)NC[C@H]1CN(c2ccc(N3CCOCC3)c(F)c2)C(=O)O1] | 8-Oct-2010 |
| 19. | Lz-NA-3 | 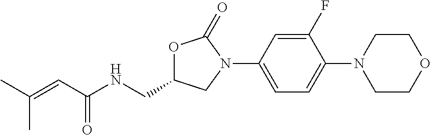 [SMILES: CC(C)=CC(=O)NC[C@H]1CN(c2ccc(N3CCOCC3)c(F)c2)C(=O)O1] | 24-Sept-2010 |
| 20. | Lz-NA-4 | 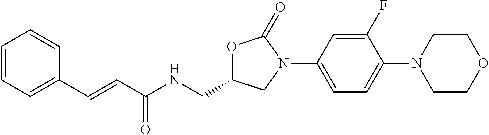 [SMILES: O=C(/C=C/c1ccccc1)NC[C@H]1CN(c2ccc(N3CCOCC3)c(F)c2)C(=O)O1] | 24-Sept-2010 |
| 21. | Lz-NA-6 | 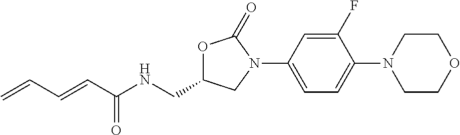 [SMILES: C=C/C=C/C(=O)NC[C@H]1CN(c2ccc(N3CCOCC3)c(F)c2)C(=O)O1] | 8-Oct-2010 |
| 22. | Lz-NA-10 | 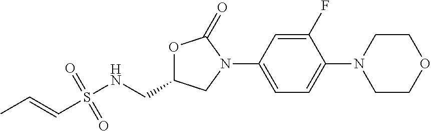 [SMILES: C/C=C/S(=O)(=O)NC[C@H]1CN(c2ccc(N3CCOCC3)c(F)c2)C(=O)O1] | 26-Nov-2010 |

### Example 1

### Evaluation of Inhibition of Tryptase Activity by Multimers

Stock solutions of recombinant human tryptase, beta, from lung (Promega: catalog number G5631, or Enzo Life Sciences: catalog number BML-SE418) were made at 30 μM, in solution with 50 μM heparin sulfate and 500 mM NaCl. Multimer tryptase inhibitor stock solutions were made at 50 mM in DMSO. Drug plates were made at 5× the final concentration in assay buffer (50 mM HEPES, 150 mM NaCl, 100 μM EDTA, pH 7.4, 0.02% Tween-20). A final concentration of 1 nM tryptase was used. When required, drugs were diluted in assay buffer immediately before use in 10-fold serial dilutions. After the indicated incubation time, the multimer-tryptase solution at 5× concentration, was diluted into assay buffer containing a final concentration of 200 μM N-tert-butoxycarbonyl-Gln-Ala-Arg-AMC HBr [AMC=7-amino-4-methylcoumarin] (Boc-Gln-Ala-Arg-AMC; Enzo Life Sciences: catalog number BML-P237) to a final volume of 50 μl in black opaque round bottom 96 well plates (Corning, catalog number 3792). The release of fluorescent AMC was immediately measured every 60 seconds over 30-60 minutes at an excitation wavelength of 367 nm, monitoring emission at 468 nm on a Spectramax M5 (Molecular Devices) microplate reader. The Softmax Pro (Molecular Devices) and Graphpad prism software were used to determine Vₘₐₓ, and concentration-response curve IC₅₀s, respectively.

### Example 2

### Evaluation of Inhibition of Ribosomal Protein Synthesis by Multimers

Monomers with the potential to form heterodimers were evaluated in an in vitro Transcription and Translation assay (TnT assay) using the commercially available E. coli S30 Extract System for Circular DNA kit (Promega Catalog # L1020) according to the manufacturers instructions with minor modifications. Monomers were tested independently to determine individual IC₅₀ values. Pairs of monomers with the potential to form heterodimers were assayed at concentrations that ranged about their individual IC25 values. Each reaction uses 2 μl (250 ng/μl) of the pBESTluc™ DNA based circular luciferase plasmid (Promega Catalog # L492A), with 4 μl of complete amino acid mix (Promega Catalog # L4461), 13 μl of S30 Premix Without Amino Acids (Promega Catalog # L512A), 5 μl of S30 Extract (Promega Catalog # L464A), monomers at the appropriate concentration, and nuclease free water in a total volume of 35 μl. Assays were carried out in Costar 96 well white round bottom plates. Assay plates were setup with a master mix consisting of S30 extract and water, followed by the addition of compound, with the final addition of a master mix consisting of the plasmid, amino acid mix, and the S30 Premix. Plates were incubated at 37° C. for one hour followed by addition of 35 μl of the Bright-Glo Luciferase Reagent (Promega Catalog # E2620). After removal of 35 μl of the reaction mixture, the luminescence was recorded immediately in the Spectramax M5 plate reader (Molecular Devices). The data was plotted to generate dose-response curves using GraphPad Prism.

### Example 3

### Synthesis of Sparsomycin Analogues

### 3-(6-methyl-2,4-dioxo-1,2,3,4-tetrahydropyrimidin-5-yl) acrylic acid

Synthesis of 3-(6-methyl-2,4-dioxo-1,2,3,4-tetrahydropyrimidin-5-yl) acrylic acid was carried out as shown in Scheme 1 below and described in the literature.

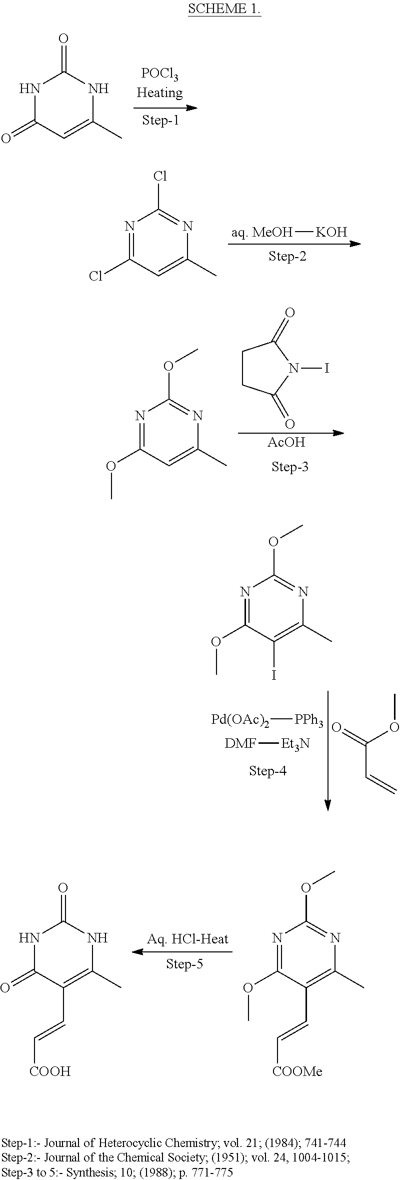

### Coupling reactions of 3-(6-methyl-2,4-dioxo-1,2,3,4-tetrahydropyrimidin-5-yl) acrylic acid

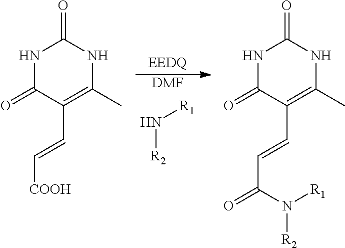

General Procedure for Coupling Reactions

100 mg (0.510 mmol) 3-(6-methyl-2,4-dioxo-1,2,3,4-tetrahydropyrimidin-5-yl)acrylic acid, desired amine (1.5 eq.), N-Ethoxycarbonyl-2-ethoxy-1,2-Dihydroquinoline (EEDQ 2 eq.) in dimethyl formamide (DMF, 5 mL) were heated to 100° C. and monitored by TLC & LCMS. After consumption of starting material the crude product was isolated either by diluting reaction mass by ethyl acetate followed by filtration of precipitated crude product, or concentrating the DMF in GeneVac® to obtain the crude product.

Dihydroxy compound (Sparso-10) was synthesized by de-methylation of corresponding dimethoxy compound (Sparso-10a) by boron tribromide in dichloromethane at room temperature.

Crude products were purified by preparative HPLC.

### Example 4

### Synthesis of N-Substituted Linezolid Derivatives with Olefinic Group

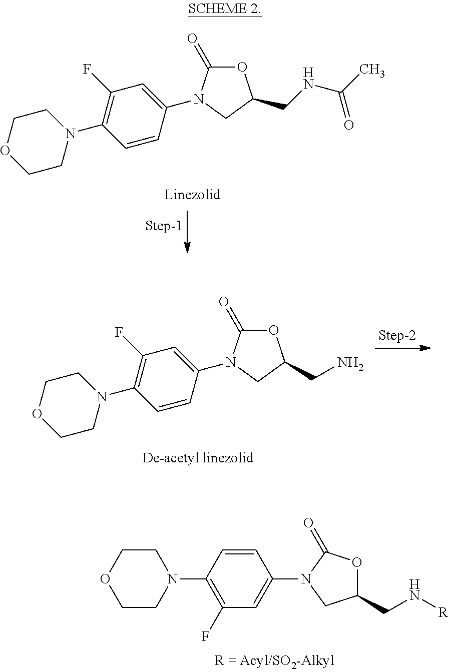

Step 1—Synthesis of De-Acetyl Linezolid

Title compound was synthesized by deacetylation of commercially available Linezolid by heating with hydroxyl amine hydrochloride (4 eq.), pyridine (35 vol) & ethanol (10 vol) in a sealed tube for 48 hrs. Solvents were then distilled and residue was diluted with dichloromethane. Inorganics were filtered and dichloromethane layer was washed with saturated sodium bicarbonate followed by brine. Concentration of dichloromethane extract resulted in crude product containing ˜20% unreacted Linezolid which was purified by crystallization from diethyl ether to get pure de-acetyl Linezolid which was sufficient pure (>95%) to use for next step.

Various N-substituted analogues were synthesized from this through coupling reactions of different carboxylic acids/sulfonyl chlorides.

Step-2—General Procedure for Coupling Reaction with Carboxylic Acids

To a stirred solution of desired carboxylic acid in 6 ml of dichloromethane, DMAP (0.5 eq.) and EDCI (1.5 eq) were added and the solution was stirred at RT for 15 mins. Deacetylated Linezolid (100 mg, 1 eq.) was then added and stirring continued at room-temperature for 4-6 hrs and reaction was monitored by LCMS & TLC (Chloroform: Methanol 9:1, Rf of product ˜0.5) till maximum starting was consumed. To the reaction mixture 5 ml of saturated sodium bicarbonate solution was added. Organic layer was separated, washed with 1N HCl (5 ml) and brine (10 ml), dried over sodium sulphate and concentrated under vacuum. Crude product was purified by reverse phase preparative HPLC and products were isolated as TFA salts.

General Procedure for Coupling Reaction with Sulfonyl Chlorides

To a stirred solution of de-acetylated Linezolid (100 mg) in dichloromethane (5 mL), triethyl amine (3 eq.) was added and the solution was cooled to 0° C. After 30 mins sulfonyl chloride (1.5 eq.) was added drop-wise. Stirring continued at room-temperature for 4-6 hrs and reaction was monitored by LCMS & TLC (Chloroform: Methanol 9:1, Rf of product ˜0.5) till maximum starting was consumed. Organic layer was then separated, washed with 1N HCl (5 ml) and Brine (5 ml), dried over sodium sulphate and concentrated under vacuum. Crude product was purified by reverse phase Preparative HPLC and products were isolated as TFA salt.

### (S)-3-(3-fluoro-4-morpholinophenyl)-5-((2-oxo-2,3-dihydro-1H-pyrrol-1-yl)methyl) oxazolidin-2-one

(S)-3-(3-fluoro-4-morpholinophenyl)-5-((2-oxo-2,3-dihydro-1H-pyrrol-1-yl)methyl) oxazolidin-2-one (Lz-NA-12) was synthesized by the reaction of 2,5-dimethoxy-2,5-dihydrofuran with de-acetyl linezolid by the procedure described in the literature for analogous transformation (J. Braz. Chem. Soc. 18, 855-859, 2007) crude product was purified by column chromatography over silica gel using Methanol(0-1%) in chloroform.

Analytical data of the compounds synthesized is as below in Table 5.

TABLE 5
ANALYTICAL DATA OF SUBSTITUTED LINEZOLID
DERIVATIVES WITH OLEFINIC GROUP

| Sr. | | | |
|---|---|---|---|
| No. | Code | Structure | Analytical data |
| 1 | Lz-NA-1 | 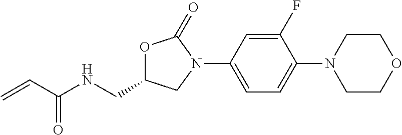 [SMILES: C=CC(=O)NC[C@H]1CN(c2ccc(N3CCOCC3)c(F)c2)C(=O)O1] | Mol. Wt: −349.35 M.I. Peak observed: 372.90(M + Na) HPLC Purity: −96.09% ¹H NMR DMSO-d6: −2.96(t, 4H), 3.50(t, 2H), 3.73(t, 4H), 4.10(t, 2H), 4.73-4.77(m, 1H), 5.60-5.62(d, 1H), 6.08-6.12(d, 1H), 6.23-6.30(m, 1H), 7.06(t, 1H), 7.16-7.18(d, 1H), 7.46- 7.50(d, 1H), 8.52(t, 1H). |
| 2 | Lz-NA-2 | 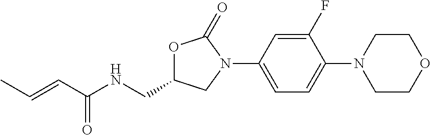 [SMILES: C/C=C/C(=O)NC[C@H]1CN(c2ccc(N3CCOCC3)c(F)c2)C(=O)O1] | Mol. Wt: −363.38 M.I. Peak observed: −364.25 HPLC Purity: −99.44 ¹H NMR DMSO-d6: −1.76- 1.78(d, 3H), 2.96(t, 4H), 3.46(t, 2H), 3.73(t, 5H), 4.08(t, 1H), 4.71- 4.73(m, 1H), 5.92-5.96(d, 1H), 6.59- 6.66(m, 1H), 7.06(t, 1H), 7.16- 7.18(d, 1H) 7.46-7.50(d, 1H), 8.30(t, 1H) |
| 3 | Lz-NA-3 | 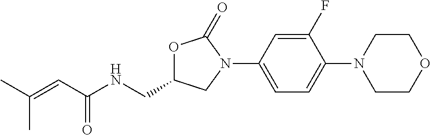 [SMILES: CC(C)=CC(=O)NC[C@H]1CN(c2ccc(N3CCOCC3)c(F)c2)C(=O)O1] | Mol. Wt: −377.41 M.I. Peak observed: 378.25 HPLC Purity: −97.16 ¹H NMR CDCl3: −1.84(s, 3H), 2.13(s, 3H), 3.06(t, 4H), 3.69(t, 2H), 3.75-3.79(m, 1H), 3.87(t, 4H), 4.00(t, 1H), 4.77-4.78(m, 1H), 5.57(s, 1H), 5.82(t, 1H), 6.94(t, 1H), 7.06-7.09(d, 1H), 7.42-7.46(d, 1H). |
| 4 | Lz-NA-4 | 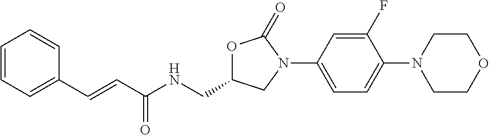 [SMILES: O=C(/C=C/c1ccccc1)NC[C@H]1CN(c2ccc(N3CCOCC3)c(F)c2)C(=O)O1] | Mol. Wt: −425.45 M.I. Peak observed: 425.95 HPLC Purity: −98.58% ¹H NMR DMSO-d6: −2.94(t, 4H), 3.56(t, 2H), 3.71-3.77(m, 5H), 4.12(t, 1H), 4.76-4.82(m, 1H), 6.664- 6.704(d, 1H), 7.05(t, 1H), 7.17- 7.19(d, 1H), 7.37- 7.56(m, 7H), 8.50(t, 1H). |
| 5 | Lz-NA-6 | 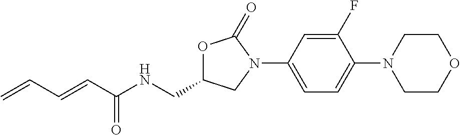 [SMILES: C=C/C=C/C(=O)NC[C@H]1CN(c2ccc(N3CCOCC3)c(F)c2)C(=O)O1] | Mol. Wt: −375.39 M.I. Peak observed: 376.30 HPLC Purity: −96.19 ¹H NMR DMSO-d6: −2.95(t, 4H), 3.51(t, 2H), 3.69-3.73(m, 5H), 4.09(t, 1H), 4.73-4.77(m, 1H), 5.41- 5.44(d, 1H), 5.57-5.61(d, 1H), 6.07- 6.11(d, 1H), 6.43-6.53(m, 1H), 7.01- 7.07(m, 2H), 7.15-7.18(d, 1H), 7.46- 7.50(d, 1H), 8.46(t, 1H). |
| 6 | Lz-NA-10 | 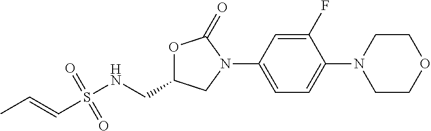 [SMILES: C/C=C/S(=O)(=O)NC[C@H]1CN(c2ccc(N3CCOCC3)c(F)c2)C(=O)O1] | Mol. Wt: −399.12 M.I. Peak observed: 400.25 HPLC Purity: −96.61 ¹H NMR DMSO-d6: −1.947-2.02(m, 3H), 2.95(t, 4H), 3.26-3.35(m, 2H), 3.73- 3.86(m, 4H), 4.09(t, 1H), 4.71- 4.73(m, 1H), 5.34-5.40(m, 2H), 5.54- 5.83(m, 1H), 7.04-7.21(m, 1H), 7.47- 7.52(d, 1H), 7.62(t, 1H), 11.68(bs, 1H) |
| 7 | Lz-NA-12 | 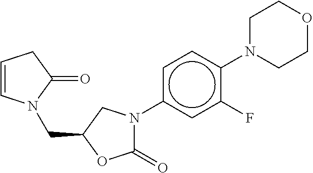 | Mol. Wt: −361.36, M.I. Peak observed: −362.25, HPLC Purity: −99.23% ¹H NMR DMSO-d6: −2.95(t, 4H), 3.51- 3.57(m, 7H), 4.10-4.16(m, 3H), 4.84- 4.90(m, 1H), 6.12-6.14(d, 1H), 7.05(t, 1H), 7.16-7.18(dd, 1H), 7.34- 7.35(d, 1H), 7.42-7.48(dd, 1H) |

### Examples 5-14

The following table contains examples 5-14.

| Sr. | | Cmpd. | Multimer | |
|---|---|---|---|---|
| No. | Cmpd. Structure | Code | Type | Target |
| 1 | 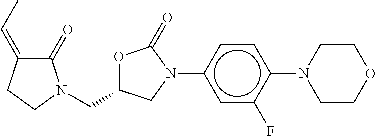 | | Hetero monomer | Ribosome |
| 2 | 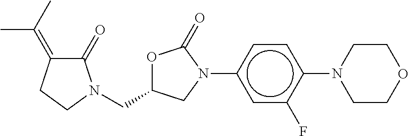 | | Hetero monomer | Ribosome |
| 3 | 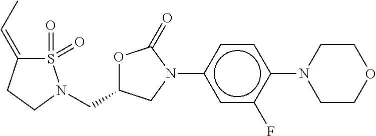 | | Hetero monomer | Ribosome |
| 4 | 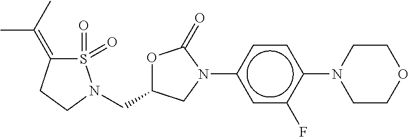 | | Hetero monomer | Ribosome |
| 5 | 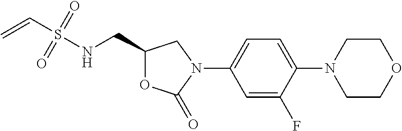 [SMILES: C=CS(=O)(=O)NC[C@H]1CN(c2ccc(N3CCOCC3)c(F)c2)C(=O)O1] | Lz-NA-7 | Hetero monomer | Ribosome |
| 6 | 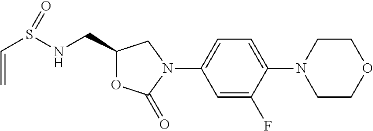 [SMILES: C=CS(=O)NC[C@H]1CN(c2ccc(N3CCOCC3)c(F)c2)C(=O)O1] | Lz-NA-9 | Hetero monomer | Ribosome |
| 7 | 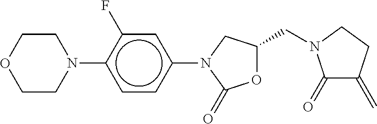 | Lz-NA-13 | Hetero monomer | Ribosome |
| 8 | 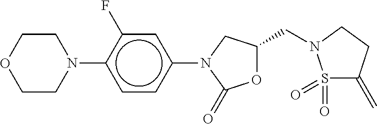 | Lz-NA-16 | Hetero monomer | Ribosome |
| 9 | 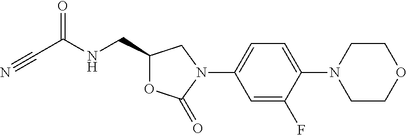 [SMILES: N#CC(=O)NC[C@H]1CN(c2ccc(N3CCOCC3)c(F)c2)C(=O)O1] | Lz-NA-5 | Hetero monomer | Ribosome |
| 10 | 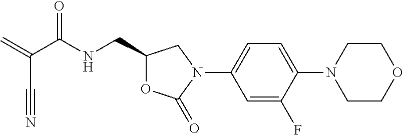 [SMILES: C=C(C#N)C(=O)NC[C@H]1CN(c2ccc(N3CCOCC3)c(F)c2)C(=O)O1] | Lz-NA-8 | Hetero monomer | Ribosome |

### EQUIVALENTS

While specific embodiments have been discussed, the above specification is illustrative and not restrictive. Many variations will become apparent to those skilled in the art upon review of this specification. The full scope of the embodiments should be determined by reference to the claims, along with their full scope of equivalents, and the specification, along with such variations.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in this specification and attached claims are approximations that may vary depending upon the desired properties sought to be obtained.

What is claimed is:

1. A first monomer capable of forming a biologically useful multimer when in contact with one, two or more second monomers in an aqueous media, wherein the first monomer is represented by the formula:
X¹—Y¹—Z¹ (Formula I)
and pharmaceutically acceptable salts, stereoisomers, metabolites and hydrates thereof, wherein
X¹ is a first ligand moiety capable of binding to and modulating a first target biomolecule;
Y¹ is absent or is a connector moiety covalently bound to X¹ and Z¹;
Z¹ is an activated π-moiety; and
the second monomer has a nucleophile moiety capable of binding with the Z¹ moiety of Formula I to form the multimer.

2. The first monomer of claim 1, wherein
Z¹ is independently selected from the group consisting of: 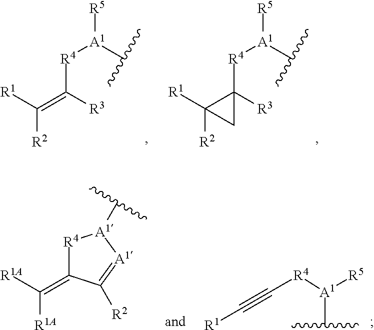
wherein
R¹ and R² are selected, independently for each occurrence, from the group consisting of hydrogen, halo, C₁₋₆alkyl, C₂₋₆alkenyl, C₂₋₆alkynyl, C₃₋₆cycloalkyl, phenyl and heteroaryl; wherein C₁₋₆alkyl, C₂₋₆alkenyl, C₂₋₆alkynyl, C₃₋₆cycloalkyl, phenyl and heteroaryl are optionally substituted with one, two, three or more substituents selected from R^{a};
R^{1A} is selected, independently for each occurrence, from the group consisting of hydrogen, halo, hydroxyl, C₁₋₆alkyl, —O—C₁₋₆alkyl, —NR³R³, C₂₋₆alkenyl, C₂₋₆alkynyl, C₃₋₆cycloalkyl, phenyl and heteroaryl; wherein C₁₋₆alkyl, C₂₋₆alkenyl, C₂₋₆alkynyl, C₃₋₆cycloalkyl, phenyl and heteroaryl are optionally substituted with one, two, three or more substituents selected from R^{a};
R^{a} is independently selected, for each occurrence, from the group consisting of halogen, hydroxyl, C₁₋₆alkyl, C₂₋₆alkenyl, C₃₋₆cycloalkyl, phenyl, heteroaryl, C₁₋₄alkoxy, C(O)C₁₋₆alkyl, C(O)C₁₋₄alkoxy, C(O)NR′R′, sulfonamide, nitro, carboxyl and cyano; wherein C₁₋₆alkyl, C₂₋₆alkenyl, C₃₋₆cycloalkyl, phenyl, heteroaryl, C₁₋₄alkoxy, C(O)C₁₋₆alkyl, C(O)C₁₋₄alkoxy and C(O)NR′R′ are optionally substituted independently, for each occurrence, with one, two, three or more substituents from the group consisting of halogen, hydroxyl, nitro and cyano;
R′ is independently selected, for each occurrence, from the group consisting of H, halogen, hydroxyl, cyano, C₁₋₄alkyl, C₂₋₆alkenyl, C₃₋₆cycloalkyl, phenyl and heteroaryl; wherein C₁₋₄alkyl, C₂₋₆alkenyl, C₃₋₆cycloalkyl, phenyl and heteroaryl are optionally substituted independently, for each occurrence, with one, two, three or more substituents from the group consisting of halogen, hydroxyl, nitro, cyano, C₁₋₄alkyl, C₂₋₆alkenyl and phenyl;
R³ is independently selected, for each occurrence, from the group consisting of hydrogen, and C₁₋₄alkyl; wherein R³ is optionally substituted with one or more substituents selected from R^{a}
R⁴ is independently selected, for each occurrence, from the group consisting of —C(O)—, —C(NR′)—, —SO₂— and —P(O)(OR′)—;
A¹ is independently selected, for each occurrence, from the group consisting of CH, N, and O;
A^{1′} is independently selected, for each occurrence, from the group consisting of CH and N;
R⁵ is independently selected, for each occurrence, from the group consisting of hydrogen and C₁₋₄alkyl; wherein if A¹ is O, there is no R⁵ substitution; or
R¹ and R⁵ may be taken with the atoms to which they are attached to form a 5-7 membered heterocycle; wherein the 5-7 membered heterocycle may optionally have 1 or 2 moieties from the group consisting of oxo, imino and sulfanylidene;
R³ and R⁵ may be taken together with the atoms to which they are attached to form a 4-7 membered heterocycle; wherein the 4-7 membered heterocycle may be substituted by one, two, three or more substituents from the group R^{a}; and wherein two R^{a} substituents may be taken together with the atoms to which they are attached to form a fused aliphatic or heteroaliphatic ring; and
the second monomer has said nucleophile moiety capable of binding with the Z¹ moiety of Formula I to form the multimer.

3. The first monomer of claim 1, wherein R⁴ is independently selected, for each occurrence, from the group consisting of —C(O)— and —SO₂—.

4. The first monomer of claim 1, wherein A¹ is N.

5. The first monomer of claim 1, wherein R¹ and R² are hydrogen.

6. The first monomer of claim 1, wherein Z¹ is represented by: 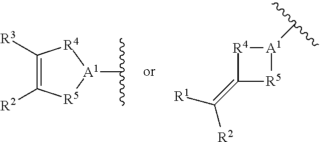
wherein
R¹ and R² are selected, independently for each occurrence, from the group consisting of hydrogen, halogen, C₁₋₆alkyl, C₂₋₆alkenyl and phenyl; wherein C₁₋₆alkyl, C₂₋₆alkenyl and phenyl are optionally substituted with one, two, three or more substituents selected from the group consisting of halogen, hydroxyl and cyano;
R³ is independently selected, for each occurrence, from the group consisting of hydrogen and C₁₋₄alkyl;
R⁴ is independently selected, for each occurrence, from the group consisting of —C(O)— and —SO₂—;
A¹ is N;
R⁵ is —C₁₋₄alkyl-; wherein if A¹ is O, there is no R⁵ substitution; or
R¹ and R⁵ may be taken together to form a 5-7 membered heterocyclic ring; or
R³ and R⁵ may be taken together to form a 5-7 membered heterocyclic ring.

7. The first monomer of claim 1, wherein Z¹ is represented by: 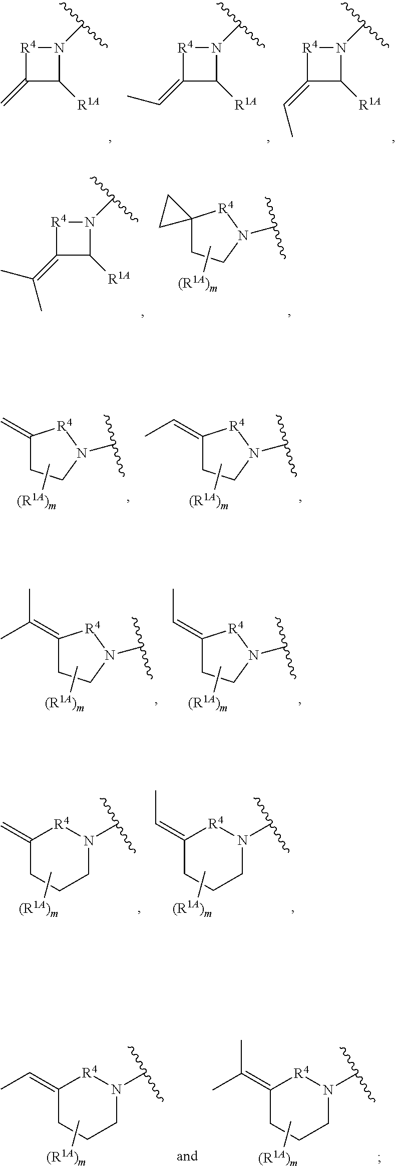
wherein
R⁴ is independently selected, for each occurrence, from the group consisting of —C(O)— and —SO₂—;
m is 0, 1, 2 or more;
R^{1A} is selected, independently for each occurrence, from the group consisting of hydrogen, halo, hydroxyl, C₁₋₆alkyl, —O—C₁₋₆alkyl, —NR^{a}R^{a}, C₂₋₆alkenyl, C₂₋₆alkynyl, C₃₋₆cycloalkyl, phenyl and heteroaryl; wherein C₁₋₆alkyl, C₂₋₆alkenyl, C₂₋₆alkynyl, C₃₋₆cycloalkyl, phenyl and heteroaryl are optionally substituted with one, two, three or more substituents selected from R^{a}; and
R^{a} is independently selected, for each occurrence, from the group consisting of halogen, hydroxyl, C₁₋₆alkyl, C₂₋₆alkenyl, C₃₋₆cycloalkyl, phenyl, heteroaryl, C₁₋₄alkoxy, C(O)C₁₋₆alkyl, C(O)C₁₋₄ alkoxy, C(O)NR′R′, sulfonamide, nitro, carboxyl and cyano; wherein C₁₋₆ alkyl, C₂₋₆alkenyl, C₃₋₆cycloalkyl, phenyl, heteroaryl, C₁₋₄alkoxy, C(O)C₁₋₆alkyl, C(O)C₁₋₄alkoxy and C(O)NR′R′ are optionally substituted independently, for each occurrence, with one, two, three or more substituents from the group consisting of halogen, hydroxyl, nitro and cyano.

8. The first monomer of claim 1, wherein Z¹ is represented by: 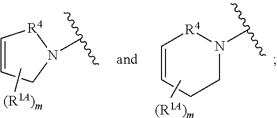
wherein
R⁴ is independently selected, for each occurrence, from the group consisting of —C(O)— and —SO₂—;
m is 0, 1, 2 or more;
R^{1A} is selected, independently for each occurrence, from the group consisting of hydrogen, halo, hydroxyl, C₁₋₆alkyl, —O—C₁₋₆alkyl, —NR^{a}R^{a}, C₂₋₆alkenyl, C₂₋₆alkynyl, C₃₋₆cycloalkyl, phenyl and heteroaryl; wherein C₁₋₆alkyl, C₂₋₆alkenyl, C₂₋₆alkynyl, C₃₋₆cycloalkyl, phenyl and heteroaryl are optionally substituted with one, two, three or more substituents selected from R^{a}; and
R^{a} is independently selected, for each occurrence, from the group consisting of halogen, hydroxyl, C₁₋₆alkyl, C₂₋₆alkenyl, C₃₋₆cycloalkyl, phenyl, heteroaryl, C₁₋₄alkoxy, C(O)C₁₋₆alkyl, C(O)C₁₋₄alkoxy, C(O)NR′R′, sulfonamide, nitro, carboxyl and cyano; wherein C₁₋₆ alkyl, C₂₋₆alkenyl, C₃₋₆cycloalkyl, phenyl, heteroaryl, C₁₋₄alkoxy, C(O)C₁₋₆alkyl, C(O)C₁₋₄alkoxy and C(O)NR′R′ are optionally substituted independently, for each occurrence, with one, two, three or more substituents from the group consisting of halogen, hydroxyl, nitro and cyano.

9. The first monomer of claim 1, wherein Z¹ is represented by: 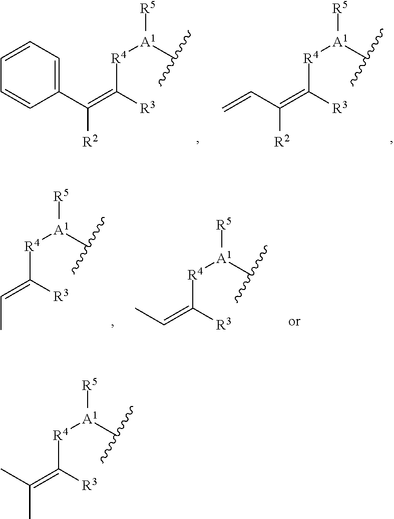
wherein
R² is selected, independently for each occurrence, from the group consisting of hydrogen and C₁₋₆alkyl; wherein C₁₋₆alkyl is optionally substituted with one, two, three or more substituents selected from the group consisting of halogen, hydroxyl and cyano;
R³ is independently selected, for each occurrence, from the group consisting of hydrogen and C₁₋₄alkyl;
R⁴ is independently selected, for each occurrence, from the group consisting of —C(O)— and —SO₂—;
A¹ is N;
R⁵ is independently selected, for each occurrence, from the group consisting of hydrogen and C₁₋₄alkyl; wherein R³ and R⁵ may be taken together to form a 5-7 membered heterocyclic ring.

10. The first monomer of claim 1, wherein Z¹ is represented by: 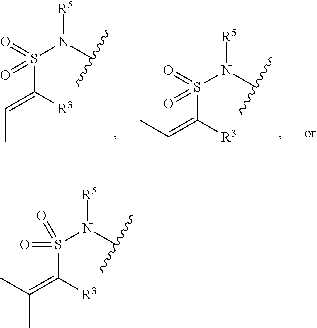
wherein
R³ is independently selected, for each occurrence, from the group consisting of hydrogen and C₁₋₄alkyl;
R⁵ is independently selected, for each occurrence, from the group consisting of hydrogen and C₁₋₄alkyl; wherein R³ and R⁵ may be taken together to form a 5-7 membered heterocyclic ring.

11. The first monomer of claim 1, wherein Z¹ is represented by: 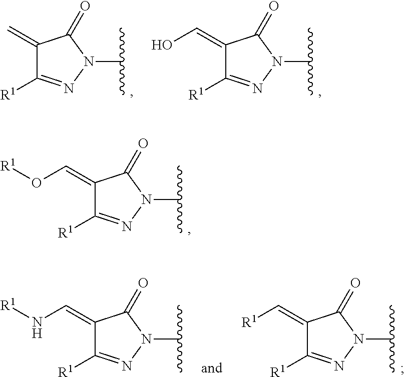
R¹ is selected, independently for each occurrence, from the group consisting of hydrogen, halo, C₁₋₆alkyl, C₂₋₆alkenyl, C₂₋₆alkynyl, C₃₋₆cycloalkyl, phenyl and heteroaryl; wherein C₁₋₆alkyl, C₂₋₆alkenyl, C₂₋₆alkynyl, C₃₋₆cycloalkyl, phenyl and heteroaryl are optionally substituted with one, two, three or more substituents selected from R^{a};
R^{a} is independently selected, for each occurrence, from the group consisting of halogen, hydroxyl, C₁₋₆alkyl, C₂₋₆alkenyl, C₃₋₆cycloalkyl, phenyl, heteroaryl, C₁₋₄alkoxy, C(O)C₁₋₆alkyl, C(O)C₁₋₄alkoxy, C(O)NR′R′, sulfonamide, nitro, carboxyl and cyano; wherein C₁₋₆alkyl, C₂₋₆alkenyl, C₃₋₆cycloalkyl, phenyl, heteroaryl, C₁₋₄alkoxy, C(O)C₁₋₆alkyl, C(O)C₁₋₄alkoxy and C(O)NR′R′ are optionally substituted independently, for each occurrence, with one, two, three or more substituents from the group consisting of halogen, hydroxyl, nitro and cyano.

12.-16. (canceled)

17. The first monomer of claim 1, wherein the second monomer may be represented by:
X²—Y²—Z² (Formula II),
and pharmaceutically acceptable salts, stereoisomers, metabolites and hydrates thereof, wherein
X² is a second ligand moiety capable of binding to and modulating a second target biomolecule;
Y² is absent or is a connector moiety covalently bound to X² and Z²;
Z² is said nucleophile moiety.

18.-29. (canceled)

30. The first monomer of claim 17, wherein Z² may be independently selected, for each occurrence, from the group consisting of: 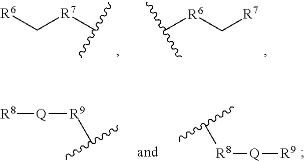
wherein
R⁶ is independently selected, for each occurrence, from the group consisting of hydrogen, C₁₋₄alkyl, phenyl, heteroaryl, C(O)NR^{b}R^{b}, and R⁷; wherein C₁₋₄alkyl, phenyl and heteroaryl are optionally substituted independently, for each occurrence, with R^{b};
R^{b} is independently selected, for each occurrence, from the group consisting of H, halogen, hydroxyl, cyano, C₁₋₄alkyl, C₂₋₆alkenyl, C₃₋₆cycloalkyl, phenyl and heteroaryl; wherein C₁₋₄alkyl, C₂₋₆alkenyl, C₃₋₆cycloalkyl, phenyl and heteroaryl are optionally substituted independently, for each occurrence, with one, two, three or more substituents from the group consisting of halogen, hydroxyl, nitro, cyano, C₁₋₄alkyl, C₂₋₆alkenyl and phenyl;
R⁷ is independently selected, for each occurrence, from the group consisting of C(O)—C₁₋₄alkyl, C(O)-phenyl, C(O)-heteroaryl, CO₂— C₁₋₄alkyl, CO₂-phenyl, CHO, cyano and nitro;
R⁸ and R⁹ are independently selected, for each occurrence, from the group consisting of hydrogen, C₁₋₄alkyl, phenyl, and heteroaryl; wherein C₁₋₄alkyl, phenyl and heteroaryl are optionally substituted independently, for each occurrence, with R_{b};
Q is independently selected, for each occurrence, from the group consisting of —O—, —S—, and —NR^{b}—.

31. (canceled)

32. A therapeutic multimer compound formed from the multimerization in an aqueous media of a first monomer represented by:
X¹—Y¹—Z¹ (Formula I)
and pharmaceutically acceptable salts, stereoisomers, metabolites and hydrates thereof,
and a second monomer represented by:
X²—Y²—Z² (Formula II)
and pharmaceutically acceptable salts, stereoisomers, metabolites and hydrates thereof.

33. The therapeutic multimer compound of claim 32, wherein
X¹ is a first ligand moiety capable of binding to and modulating a first target biomolecule;
Y¹ is absent or is a connector moiety covalently bound to X¹ and Z¹;
Z¹ is selected from the group consisting of: 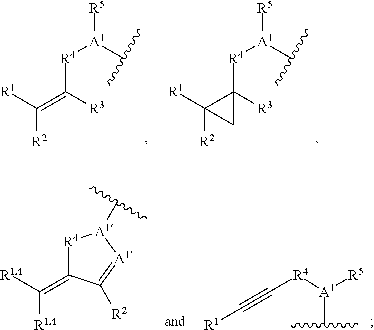
wherein
R¹ and R² are selected, independently for each occurrence, from the group consisting of hydrogen, halo, C₁₋₆alkyl, C₂₋₆alkenyl, C₂₋₆alkynyl, C₃₋₆cycloalkyl, phenyl and heteroaryl; wherein C₁₋₆alkyl, C₂₋₆alkenyl, C₂₋₆alkynyl, C₃₋₆cycloalkyl, phenyl and heteroaryl are optionally substituted with one, two, three or more substituents selected from R^{a};
R^{1A} is selected, independently for each occurrence, from the group consisting of hydrogen, halo, hydroxyl, C₁₋₆alkyl, —O—C₁₋₆alkyl, —NR³R³, C₂₋₆alkenyl, C₂₋₆alkynyl, C₃₋₆cycloalkyl, phenyl and heteroaryl; wherein C₁₋₆alkyl, C₂₋₆alkenyl, C₂₋₆alkynyl, C₃₋₆cycloalkyl, phenyl and heteroaryl are optionally substituted with one, two, three or more substituents selected from R^{a};
R^{a} is independently selected, for each occurrence, from the group consisting of halogen, hydroxyl, C₁₋₆alkyl, C₂₋₆alkenyl, C₃₋₆cycloalkyl, phenyl, heteroaryl, C₁₋₄alkoxy, C(O)C₁₋₆alkyl, C(O)C₁₋₄alkoxy, C(O)NR′R′, sulfonamide, nitro, carboxyl and cyano; wherein C₁₋₆alkyl, C₂₋₆alkenyl, C₃₋₆cycloalkyl, phenyl, heteroaryl, C₁₋₄alkoxy, C(O)C₁₋₆alkyl, C(O)C₁₋₄alkoxy and C(O)NR′R′ are optionally substituted independently, for each occurrence, with one, two, three or more substituents from the group consisting of halogen, hydroxyl, nitro and cyano;
R′ is independently selected, for each occurrence, from the group consisting of H, halogen, hydroxyl, cyano, C₁₋₄alkyl, C₂₋₆alkenyl, C₃₋₆cycloalkyl, phenyl and heteroaryl; wherein C₁₋₄alkyl, C₂₋₆alkenyl, C₃₋₆cycloalkyl, phenyl and heteroaryl are optionally substituted independently, for each occurrence, with one, two, three or more substituents from the group consisting of halogen, hydroxyl, nitro, cyano, C₁₋₄alkyl, C₂₋₆alkenyl and phenyl;
R³ is independently selected, for each occurrence, from the group consisting of hydrogen, and C₁₋₄alkyl; wherein R³ is optionally substituted with one or more substituents selected from R^{a}
R⁴ is independently selected, for each occurrence, from the group consisting of —C(O)—, —C(NR′)—, —SO₂— and —P(O)(OR′)—;
A¹ is independently selected, for each occurrence, from the group consisting of CH, N, and O;
A^{1′} is independently selected, for each occurrence, from the group consisting of CH and N;
R⁵ is independently selected, for each occurrence, from the group consisting of hydrogen and C₁₋₄alkyl; wherein if A¹ is O, there is no R⁵ substitution; or
R¹ and R⁵ may be taken with the atoms to which they are attached to form a 5-7 membered heterocycle; wherein the 5-7 membered heterocycle may optionally have 1 or 2 moieties from the group consisting of oxo, imino and sulfanylidene;
R³ and R⁵ may be taken together with the atoms to which they are attached to form a 4-7 membered heterocycle; wherein the 4-7 membered heterocycle may be substituted by one, two, three or more substituents from the group R^{a}; and wherein two R^{a} substituents may be taken together with the atoms to which they are attached to form a fused aliphatic or heteroaliphatic ring; and
X² is a second ligand moiety capable of binding to and modulating a second target biomolecule;
Y² is absent or is a connector moiety covalently bound to X² and Z²;
Z² is selected from the group consisting of: 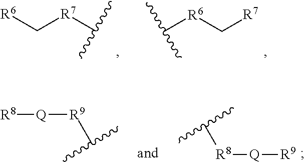
wherein
R⁶ is independently selected, for each occurrence, from the group consisting of hydrogen, C₁₋₄alkyl, phenyl, heteroaryl, C(O)NR^{b}R^{b}, and R⁷; wherein C₁₋₄alkyl, phenyl and heteroaryl are optionally substituted independently, for each occurrence, with R_{b};
R^{b} is independently selected, for each occurrence, from the group consisting of H, halogen, hydroxyl, cyano, C₁₋₄alkyl, C₂₋₆alkenyl, C₃₋₆cycloalkyl, phenyl and heteroaryl; wherein C₁₋₄alkyl, C₂₋₆alkenyl, C₃₋₆cycloalkyl, phenyl and heteroaryl are optionally substituted independently, for each occurrence, with one, two, three or more substituents from the group consisting of halogen, hydroxyl, nitro, cyano, C₁₋₄alkyl, C₂₋₆alkenyl and phenyl;
R⁷ is independently selected, for each occurrence, from the group consisting of C(O)—C₁₋₄alkyl, C(O)-phenyl, C(O)-heteroaryl, CO₂— C₁₋₄alkyl, CO₂-phenyl, CHO, cyano and nitro;
R⁸ and R⁹ are independently selected, for each occurrence, from the group consisting of hydrogen, C₁₋₄alkyl, phenyl, and heteroaryl; wherein C₁₋₄alkyl, phenyl and heteroaryl are optionally substituted independently, for each occurrence, with R_{b};
Q is independently selected, for each occurrence, from the group consisting of —O—, —S—, and —NR^{b}—.

34. The therapeutic multimer compound of claim 32, wherein formation of the multimer is substantially irreversible in an aqueous media.

35. The therapeutic multimer compound of claim 32, wherein formation of the multimer is substantially reversible in an aqueous media.

36. The therapeutic multimer compound of claim 32, wherein X¹ and X² are the same.

37. The therapeutic multimer compound of claim 32, wherein X¹ and X² are different.

38. A method of modulating two or more target biomolecules substantially simultaneously comprising:
contacting an aqueous composition comprising said bimolecular target with a first monomer represented by:
X¹—Y¹—Z¹ (Formula I)
and pharmaceutically acceptable salts, stereoisomers, metabolites and hydrates thereof, wherein
X¹ is a first ligand moiety capable of binding to and modulating a first target biomolecule; and
a second monomer represented by:
X²—Y²—Z² (Formula II),
and pharmaceutically acceptable salts, stereoisomers, metabolites and hydrates thereof, wherein
X₂ is a ligand moiety capable of binding to and modulating a second target biomolecule;
wherein upon contact with the aqueous composition, said first monomer and said second monomer forms a multimer that binds to the first target biomolecule and the second target biomolecule.

39.-46. (canceled)
